(19) United States
(12) Patent Application Publication
(10) Pub. No.: US 2024/0325461 A1
(43) Pub. Date: Oct. 3, 2024

(54) COMBINATION THERAPIES FOR THE TREATMENT OF DISEASES

(71) Applicant: Research Development Foundation, Carson City, NV (US)

(72) Inventors: Mirko TRAJKOVSKI, Geneva (CH); Melis ULGEN, Geneva (CH)

(73) Assignee: Research Development Foundation, Carson City, NV (US)

(21) Appl. No.: 18/619,514

(22) Filed: Mar. 28, 2024

Related U.S. Application Data
(60) Provisional application No. 63/492,820, filed on Mar. 29, 2023.

(51) Int. Cl. A61K 35/741; A61K 31/663; A61K 35/00; A61K 35/747; A61P 3/04; A61P 3/10; A61P 19/10

(52) U.S. Cl. CPC A61K 35/741; A61K 31/663; A61K 35/747; A61P 3/04; A61P 3/10; A61P 19/10; A61K 2035/115

(57) ABSTRACT

Disclosed are combination therapies for the treatment of diseases including obesity and osteoporosis. In some embodiments, an inactivated Parabacteroides goldsteinii is enterically administered to a subject, such as a human patient, in combination with a bisphosphonate such as alendronate to treat the metabolic disease or disorder. Related pharmaceutical and probiotic compositions and methods are provided.

This application claims benefit of priority to U.S. Provisional Application Ser. No. 63/492,820 filed Mar. 29, 2023, the entire contents of which is hereby incorporated by reference.

### BACKGROUND OF THE INVENTION

### 1. Field of the Invention

The present invention relates generally to the fields of biology and medicine. More particularly, it concerns methods and compositions for the treatment of disease, such as metabolic diseases and disorders.

### 2. Description of Related Art

Obesity, type 2 diabetes, and osteoporosis remain significant clinical problems. Obesity is a relatively common medical condition that can result in very serious adverse health consequences, such as heart disease, diabetes, high blood pressure, and certain cancers. Type 2 diabetes is characterized by resistance to insulin, and the disease can lead to several very serious health consequences including, e.g., neuropathies, skin problems, and/or kidney damage. Post-menopausal weight gain can also contribute to health problems in many individuals. The rates of obesity and type 2 diabetes are relatively high. For example, the number of people with obesity or type 2 diabetes in the U.S. alone are currently over 70 million people and over 30 million people, respectively.

The gut of a healthy mammalian subject is inhabited with microbiota that can affect human health. In some cases, the gut microbiome may play a role in the development of obesity, but the mechanism by which this may happen is complicated and not well understood (e.g., see Davis et al., 2016). For example, Lactobacillus reuteri has been observed to be increased in individuals with obesity (Million et al., 2012). Clearly there is a need for new treatments for obesity and type 2 diabetes.

Osteoporosis is the most prevalent metabolic bone disorder, characterized by low bone mass and microarchitectural deterioration (Sozen et al., 2017). Patients with osteoporosis have fragile bones and are vulnerable to fractures. The most common type of primary osteoporosis is due to the post-menopausal estrogen deficiency, reflected in a higher incidence of osteoporosis in women (Reginster and Burlet, 2006). Although some therapies have been developed for the treatment of osteoporosis, some patients do not respond to these therapies, and there is a need for new and improved therapies.

### SUMMARY OF THE INVENTION

The present disclosure is based, in part, on the discovery that administration of (i) a bisphosphonate (e.g., alendronate) and (ii) live or inactivated Parabacteroides goldsteinii (e.g., heat-inactivated Parabacteroides goldsteinii) delivered to the gastrointestinal tract of a subject, can be used to synergistically treat metabolic disorders and/or bone diseases. The combination therapies described herein can be used for the treatment of a variety of metabolic or bone diseases including obesity, fatty liver disease, dyslipidemia, type II diabetes, osteoporosis, osteopenia, Paget's disease, post-menopausal weight gain, post-menopausal glucose intolerance, and post-menopausal osteoporosis. It is anticipated that synergistic effects resulting from the combination therapy with inactivated P. goldsteinii may enable lower dosages of the bisphosphonate (e.g., alendronate) to be administered to a patient, thereby reducing or eliminating one or more side effect associated with the bisphosphonate.

As shown in the below examples, alendronate alone did not result in any weight loss in a curative in vivo model of post-menopausal weight gain; however, when administered to mice in combination with inactivated Parabacteroides goldsteinii, increased weight loss was observed even beyond the inactivated P. goldsteinii (e.g., FIGS. 2A-B, FIGS. 3A-B). Inactivated P. goldsteinii and alendronate resulted in at least additive or synergistic improvements in bone density in vivo using a mouse model of post-menopausal osteoporosis. Insulin sensitivity was also unaffected by alendronate or inactivated P. goldsteinii, but when these agents were co-administered to the mouse model of post-menopausal osteoporosis, then improvements were observed (e.g., FIGS. 5A-B), and this combination therapy also improved glucose tolerance (FIGS. 6A-B). Further, these results were observed after onset of the disease and in some experiments improvements in the physical properties of bone (e.g., ultimate stress and elastic energy) were observed that even exceeded control mice. When administered individually, heat-inactivated P. goldsteinii or alendronate did not statistically improve elastic energy of femurs in an in vivo curative mouse model of post-menopausal osteoporosis; however, when heat-inactivated P. goldsteinii or alendronate were co-administered, elastic energy of femurs in mice was synergistically improved to a level beyond even the control mice. Synergistic effect of Alendronate and heat-inactivated P. goldsteinii (HI P. goldsteinii) in reverting ovariectomy-induced bone, using parameters of ultimate stress, yield point, and elastic energy, are shown in FIG. 7, FIG. 9, and FIG. 10. The combination therapy also improved bone stiffness (FIG. 10). The combination therapies provided herein may also be used to improve insulin sensitivity or glucose intolerance in a mammalian subject such as a human (e.g., FIGS. 5A-B, FIGS. 6A-B). It was further observed that living P. goldsteinii or alendronate can also be used to achieve synergistic improvements in metabolic and bone parameters using a mouse model of osteoporosis. A synergistic effect of alendronate and P. goldsteinii (HI and Live form) was observed for preventing ovariectomy-induced trabecular bone remodeling (FIGS. 11A-B). Synergistic effects of alendronate and P. goldsteinii (HI or live) were observed for preventing ovariectomy-induced bone remodeling, as observed in trabecular thickness (FIG. 12A), cortical thickness of femur (FIG. 12B), trabecular number (FIG. 13A), trabecular connectivity density (FIG. 13B), trabecular spacing (FIG. 14A), trabecular mineralization (FIG. 14B), ultimate strength (FIG. 15A), yield point (FIG. 15B), elastic energy (FIG. 15C), and stiffness (FIG. 15D). HI P. goldsteinii and alendronate synergistically prevented ovariectomy-induced increases in body weight (FIGS. 16A-B). HI P. goldsteinii and alendronate prevented ovariectomy-induced decreases in glucose tolerance (FIGS. 17A-B). Bisphosphonates (e.g., alendronate) initially prevent bone resorption, but this effect then leads to the problem of a compensatory decrease in bone formation, since resorption and formation are coupled (Jensen et. al, Bone, 2021; Shimizu et. al, J Dent Res, 2012; Iwata et. al, Bone, 2006). Administration of either HI or live Parabacteroides goldsteinii were surprisingly observed to prevent this Alendronate-induced reduction in P1NP levels (FIGS. 18A-B). Both live and HI P. goldsteinii exerted anti-osteoporotic effects and may be used alone or together in combination with a bisphosphonate such as alendronate, optionally further in combination with one or more additional drugs (e.g., for the prevention or treatment of osteoporosis). Without wishing to be bound by any theory, data is presented supporting the idea that live and HI P. goldsteinii may exert biological functions through affecting osteoblasts. The HI or live P. goldsteinii may preferably be administered orally or mucosally to the gastrointestinal system of a subject. The combination therapies can optionally be administered to a subject in combination with additional bacteria (e.g., Lactobacillus gasseri or Lactobacillus reuteri), spermine, spermidine, or an additional diabetes medication or other therapeutic. It is anticipated that these synergistic effects may enable lower dosages of a bisphosphonate (e.g., alendronate) to be administered to a patient, when administered in combination with inactivated P. goldsteinii (e.g., heat-inactivated P. goldsteinii), to treat a metabolic disease or disorder (e.g., obesity) or a bone disease (e.g., osteoporosis), thereby reducing or eliminating one or more side effect associated with the bisphosphonate (e.g., a side effect associated with the dosage of alendronate or risedronate approved by the FDA as a monotherapy for treatment of osteoporosis).

An aspect of the present disclosure relates to a method of treating a disease in a mammalian subject, comprising administering to the mammalian subject a therapeutically relevant amount of: (i) a composition comprising a Parabacteroides goldsteinii, wherein the composition is delivered to the gastrointestinal system of the mammalian subject, and (ii) a bisphosphonate; wherein the disease is metabolic disease or disorder or a bone disease.

The bisphosphonate may be alendronate, risedronate, ibandronate, zoledronic acid, denosumab, raloxifene, or bazedoxifene. In some embodiments, the bisphosphonate is alendronate. In some embodiments, about 10-80 or 35-70 mg per week of alendronate or about 5-10 mg per day of alendronate is administered to the subject. In some embodiments, less than 35 mg per week of alendronate or less than 5 mg per day of alendronate is administered to the subject. In some embodiments, about 10-30 mg per week of alendronate or about 1-4 mg per day of alendronate is administered to the subject. The alendronate may be administered orally, intravenously, intraperitoneally, or subcutaneously. In some embodiments, the Parabacteroides goldsteinii is living or is not inactivated. The Parabacteroides goldsteinii may be inactivated. In some embodiments, the Parabacteroides goldsteinii is heat-inactivated. In some embodiments, the Parabacteroides goldsteinii have been inactivated by heating to about 95-105° C. for about 10-20 min, or by heating to about 100° C. for about 15 min. In some embodiments, the inactivated Parabacteroides goldsteinii has been inactivated via exposure to a peroxide (e.g., hydrogen peroxide, hydrogen peroxide vapor, or via exposure to radiation or ionizing radiation. The ionizing radiation may comprise or consist of light having a wavelength of about 400-420 nm. In some embodiments, the inactivated Parabacteroides goldsteinii has been inactivated via exposure to air plasma, ultrasound under pressure, an alcohol (e.g., ethanol), high hydrostatic pressure (HHP), or pulsed electric field (PEF). The composition may comprise extracellular vesicles from Parabacteroides goldsteinii. The composition may comprise about 1×10⁸-1×10¹³ or about 1×10⁹-1×10¹⁰ CFU of the inactivated Parabacteroides goldsteinii. In some embodiments, the subject is administered from about 0.25*10⁹ to about 12*10¹⁰ cells/kg body weight of the subject of the inactivated Parabacteroides goldsteinii. In some embodiments, the subject is administered from about 0.97-1.62*10⁹ or about 6.8-11.3*10¹⁰ cells/kg body weight of the subject of the inactivated Parabacteroides goldsteinii. The inactivated Parabacteroides goldsteinii may be heat-inactivated Parabacteroides goldsteinii. The inactivated Parabacteroides goldsteinii may be administered to the subject once per day or once every two days. A variety of dosages may be administered to the mammalian subject including, e.g., 0.97-1.62*10⁹ cells/kg body weight of the subject and other dosages and administration schedules (e.g., for mice, 200 μ1 bacteria with concentration of 1,5-2.5 OD/ml at 600 nm corresponding to 2.4-4*10⁸ cells (200 μ1 of 1.5-2.5 OD/ml), equivalent to 1.2-2*10¹⁰ cells/kg mouse body weight (BW) can be administered every other day; for humans, about 0.97-1.62*10⁹ cells/kg BW, or 6.8-11.3*10¹⁰ cells for 70 kg subject can be administered every other day (alternatively per day); a range of 10⁹-10¹⁰ per day; or ranges between 10⁸-10¹³ cells per day or every other day or 10⁸-10¹⁵ per week or per month can be administered). The composition may further comprise Lactobacillus gasseri, Lactobacillus reuteri, or Akkermansia muciniphila. In some embodiments, the composition is further defined as a pharmaceutical composition or a probiotic composition. The composition may further comprise Lactobacillus gasseri and Lactobacillus reuteri. The composition may further comprise extracellular vesicles from Lactobacillus gasseri or Lactobacillus reuteri. The pharmaceutical or probiotic composition may be administered orally, colonically, via enema, via an orogastric tube, or via a nasogastric tube. The inactivated Parabacteroides goldsteinii or vesicles from Parabacteroides goldsteinii may be comprised in a pharmaceutical or probiotic composition that is resistant to degradation in the stomach but releases bacteria in the small intestine and/or large intestine of the subject. The pharmaceutical or probiotic composition may comprise an enteric coating, chitosan-alginate beads, or a hydrogel. The enteric coating may be a fatty acid, a wax, a shellac, a plastic such as a phthalate, CAP, CAT, PVAP, HPMCP, or a plant fiber. In some embodiments, the pharmaceutical or probiotic composition does not comprise an enteric coating. The pharmaceutical or probiotic composition may be a tablet or capsule. In some embodiments, the subject is a human (e.g., a postmenopausal woman). The metabolic disease or disorder may be obesity, type 2 diabetes, fatty liver disease, glucose intolerance, insulin resistance, post-menopausal weight gain, post-menopausal glucose intolerance, or dyslipidemia. In some embodiments, the metabolic disease or disorder is obesity, fatty liver disease, or nonalcoholic fatty liver disease (NAFLD). In some embodiments, the subject does not have diabetes. In some embodiments, the bone disease is osteoporosis, osteomalacia, osteolysis, osteochondrodysplasias, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, or Paget's disease. In some embodiments, the bone disease is osteoporosis. The method may further comprise administering an estrogen therapy to the subject. In some embodiments, the microbiota in the composition has been purified or cultured. The method may further comprise enterically administering spermine and/or spermidine to the subject. The method may comprise enterically administering both spermine and spermidine to the subject. The method may comprise administering about 1-50 mg per kg body weight per day spermine to the subject. The method may comprise administering about 1-50 mg per kg body weight per day spermidine to the subject. The composition may comprise the spermine and/or spermidine. In some embodiments, the composition comprises both spermine and spermidine. In some embodiments, Parabacteroides goldsteinii are cultured or expanded in a medium comprising spermidine and/or spermine prior to inactivation. The medium may comprise about 0.1-6 mM spermidine and/or about 0.1-6 mM spermine. In some embodiments, the subject is administered antibiotics and exposed to an environment of about 25-50° C., more preferably about 32-35° C. for at least about 15 minutes.

Another aspect of the present disclosure relates to a pharmaceutical or probiotic composition comprising: (i) Parabacteroides goldsteinii, the growth medium of Parabacteroides goldsteinii, or vesicles from Parabacteroides goldsteinii, and (ii) a biphosphate; wherein the composition is formulated for delivery to the gastrointestinal system. The Parabacteroides goldsteinii may preferably be inactivated. The Parabacteroides goldsteinii may be living or not inactivated. As shown in the examples, both living and inactivated Parabacteroides goldsteinii can each be administered in combination with a bisphosphonate to achieve synergistic improvements in a bone and/or metabolic disease. The biphosphate may be alendronate, risedronate, ibandronate, zoledronic acid, denosumab, raloxifene, or bazedoxifene. In some embodiments the bisphosphonate is alendronate. The composition may comprise about 1-75 mg or about 5-70 mg of alendronate, or 1, 2, 3, 4, 5, 6, 7, 8, 9, 10, 15, 20, 25, 30, 35, 40, 45, 50, 55, 60, 65, 70, or 75 mg of alendronate, or any range derivable therein. In some embodiments, the composition comprises less than 5 mg or about 1-4 mg of alendronate. The composition may comprise about 1×10⁸-1×10¹³ or about 1×10⁹-1×10¹⁰ cfu of the inactivated Parabacteroides goldsteinii. The composition may comprise from about 6.5-11.5*10¹⁰ cells (e.g., 6.8-11.3*10¹⁰ cells) of heat-inactivated Parabacteroides goldsteinii. The inactivated Parabacteroides goldsteinii may be heat-inactivated Parabacteroides goldsteinii. The composition may further comprise Lactobacillus gasseri or Lactobacillus reuteri. The composition may further comprise extracellular vesicles from Lactobacillus gasseri or extracellular vesicles from Lactobacillus reuteri. The pharmaceutical or probiotic composition may be formulated for oral, colonic, enema, orogastric, or nasogastric administration. In some embodiments, the pharmaceutical or probiotic composition is resistant to degradation in the stomach but releases bacteria in the small intestine and/or large intestine of the subject. The pharmaceutical or probiotic composition may comprise an enteric coating, chitosan-alginate beads, or a hydrogel. The enteric coating may be a fatty acid, a wax, a shellac, a plastic such as a phthalate, CAP, CAT, PVAP, HPMCP, or a plant fiber. In some embodiments, the pharmaceutical or probiotic composition does not comprise an enteric coating. In some embodiments, the pharmaceutical or probiotic composition is a tablet or capsule. The pharmaceutical or probiotic composition may further comprise spermine or spermidine. The pharmaceutical or probiotic composition may comprise 1-3500 mg spermine. The pharmaceutical or probiotic composition may comprise 1-3500 mg of spermidine. In some embodiments, the pharmaceutical or probiotic composition comprises both spermine and spermidine. In some embodiments, the Parabacteroides goldsteinii has been inactivated via exposure to a peroxide, ionizing radiation, heat, air plasma, ultrasound under pressure, an alcohol, high hydrostatic pressure (HHP), or pulsed electric field (PEF). In some embodiments, the Parabacteroides goldsteinii has been inactivated via exposure to a peroxide, ionizing radiation, or heat. In some embodiments, the composition is for use in treating a metabolic disease or disorder in a mammalian subject. The metabolic disease or disorder may be obesity, type 2 diabetes, fatty liver disease, a nonalcoholic fatty liver disease (NAFLD), insulin resistance, or dyslipidemia. In some embodiments, the subject is a human, such as for example a postmenopausal woman.

Lactobacillus gasseri is a species of bacteria that has been identified as part of the vaginal flora and has been found in the lower digestive systems of women. Particular strains of Lactobacillus gasseri that may be used to treat a metabolic disease or disorder in a mammalian subject can include DSM 20077, DSM 107525, DSM 20243, DSM 20604, ATCC® 3332, ATCC® 2960, ATCC® BAA-2841, ATCC® PTA4483, ATCC® PTA4481, ATCC® PTA4484, ATCC® PTA4480, and/or ATCC® PTA44 79. A variety of amounts of Lactobacillus gasseri may be administered to a mammalian subject (e.g., a human) to treat a metabolic disease or disorder as described herein (e.g., obesity, type 2 diabetes, fatty liver, etc.). For example, in some embodiments from about 1×10⁸ to about 1×10¹³ cfu of Lactobacillus gasseri can be administered to a mammalian subject, such as a human, to treat the metabolic disease or disorder.

Lactobacillus reuteri is a species of bacteria that has been found in the intestinal tract of healthy mammals. Particular strains of Lactobacillus reuteri that may be used to treat a metabolic disease or disorder in a mammalian subject include DSM 100191, DSM 100192, DSM 17509, DSM 20015, DSM 20016, DSM 20053, DSM 20056, DSM 28673, DSM 32035, ATCC® BAA-2837™, ATCC®55148, ATCC®53608, ATCC® 23272, ATCC® 23272D5, and/or ATCC® PTA6475. A variety of amounts of Lactobacillus reuteri may be administered to a mammalian subject (e.g., a human) to treat a metabolic disease or disorder as described herein (e.g., obesity, type 2 diabetes, fatty liver, etc.). For example, in some embodiments from about 1×10⁸ to about 1×10¹³ cfu of Lactobacillus reuteri can be administered to a mammalian subject, such as a human, to treat the metabolic disease or disorder.

Parabacteroides goldsteinii is a gram-negative, obligately anaerobic non-spore-forming and non-motile bacterium that has been isolated from human blood. Particular strains of Parabacteroides goldsteinii that may be used to treat a metabolic disease or disorder in a mammalian subject include DSM 19448 and/or DSM 29187. A variety of amounts of Parabacteroides goldsteinii may be administered to a mammalian subject (e.g., a human) to treat a metabolic disease or disorder as described herein (e.g., obesity, type 2 diabetes, fatty liver, etc.). For example, in some embodiments from about 1×10⁸ to about 1×10¹³ cfu of inactivated Parabacteroides goldsteinii can be administered to a mammalian subject, such as a human, to treat the metabolic disease or disorder. As shown in the below examples, heat-inactivated Parabacteroides goldsteinii can be administered to treat a metabolic disease or disorder. Methods of heat inactivation that may be used to prepare heat-inactivated Parabacteroides goldsteinii are well known and include heating up the bacteria to about 100° C. for about 15 minutes (Wu et al., 2019). Parabacteroides goldsteinii might also be conserved by freezing or by dehydration. A variety of methods can be used to generate inactivated Parabacteroides goldsteinii. For example, in some embodiments, the bacteria may be irradiated or killed by radiation, exposure to ethanol, or autoclaving (e.g., as described in Lin et al., 2015.). In some embodiments, the Parabacteroides goldsteinii can be inactivated by exposure to light comprising or consisting of light having a wavelength of about 405 nm (e.g., Maclean et al., 2009). The inactivated Parabacteroides goldsteinii can be generated via exposure to air plasma, such as a direct-current, cold-atmospheric-pressure air plasma microjet (e.g., Tian et al., 2010). The inactivated Parabacteroides goldsteinii can be generated via exposure to hydrogen peroxide or hydrogen peroxide vapor (e.g., Malik et al., 2013; Erttmann et al., 2019; Grigoryan et al., UDC 579.67). The inactivated Parabacteroides goldsteinii can be generated via exposure to ionizing irradiation, ultrasound under pressure, high hydrostatic pressure (HHP), and/or pulsed electric field (PEF) (e.g., Manas, et al., 2005). In some preferred embodiments, Parabacteroides goldsteinii are killed using heat inactivation.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

Other objects feature and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Curative model of osteoporosis.

FIGS. 2A-B: (FIG. 2A) Body weight gain of ovariectomized female mice starting 2 months after the surgery to address the curative effects of the treatment until day 29 after starting the administration. Mice were administered with the respective treatment starting at day 0, which is 2 months flowing the surgery. The values are normalized to the initial body weight at the start of the treatment. (FIG. 2B) Area under the curve of mice as in (A) until day 29 after starting the treatment. (n=6-8 per group, data points show mean values+STD). Significance is calculated using Tukey's multiple comparison One-way Anova test: * p≤0.05; ** p≤0.01; *** p≤0.001; **** p≤0.0001.

FIGS. 3A-B: (FIG. 3A) Body weight gain of ovariectomized female mice starting 2 months after the surgery to address the curative effects of the treatment until day 60 after starting the administration. Mice were administered with the respective treatment starting at day 0, which is 2 months flowing the surgery. The values are normalized to the initial body weight at the start of the treatment. (FIG. 3B) Area under the curve of mice as in (FIG. 3A) until day 60 after starting the treatment. (n=6-8 per group, data points show mean values+SEM). Significance is calculated using Tukey's multiple comparison One-way Anova test: * p≤0.05; ** p≤0.01; *** p≤0.001; **** p≤0.0001.

FIG. 4: Absolute body weights of ovariectomized female mice as in FIGS. 3A-B, starting 2 months after the surgery to address the curative effects of the treatment until day 60, after starting the administration. Mice were administered with the respective treatment starting at day 0, which is 2 months flowing the surgery.

FIGS. 5A-B: (FIG. 5A) Insulin sensitivity (normalized values) measured by intraperitoneal insulin tolerance test of ovariectomized female mice as in FIGS. 3A-B, administered with the respective treatment starting 2 months flowing the surgery, considered as day 0. Test is performed on day 52 after staring the administration. (FIG. 5B) Area under the curve of mice as in (A). (n=6-8 per group, data points show mean values+SEM). Significance is calculated using Dunnet's multiple comparison One-way Anova test: * p≤0.05; ** p≤0.01; *** p≤0.001; **** p≤0.0001.

FIGS. 6A-B: (FIG. 6A) Glucose tolerance measured by intraperitoneal glucose tolerance test of ovariectomized female mice as in FIGS. 3A-B, administered with the respective treatment starting 2 months flowing the surgery, considered as day 0. Test is performed on day 52 after staring the administration. (FIG. 6B) Area under the curve of mice as in (A). (n=6-8 per group, data points show mean values+SEM). Significance is calculated using Dunnet's multiple comparison One-way Anova test: * p≤0.05; ** p≤0.01; *** p≤0.001; **** p≤0.0001.

FIG. 7: Synergistic effect of Alendronate and HI P. goldsteinii in reverting ovariectomy-induced bone (femur midshaft) weakening revealed through biomechanical analysis of Ultimate stress and Yield point. Ultimate stress and Yield point measured by 3-point bending test in midshaft of femur from ovariectomized female mice as in FIG. 2, administered with the respective treatment starting 2 months flowing the surgery. Test is performed postmortem, and mice are sacrificed on day 60 after staring the administration. (n=6-10 per group, data points show mean values+SD). Significance is calculated using Dunnet's multiple comparison One-way Anova test: * p≤0.05; ** p≤0.01.

FIG. 8: Elastic energy, and Stiffness measured by 3-point bending test in midshaft of femur from ovariectomized female mice as in FIGS. 2YA-B, administered with the respective treatment starting 2 months flowing the surgery. Test is performed postmortem, and mice are sacrificed on day 60 after staring the administration. (n=6-10 per group, data points show mean values+SD). Significance is calculated using Dunnet's multiple comparison One-way Anova test).

FIG. 9: Synergistic effect of Alendronate and HI P. goldsteinii in reverting ovariectomy-induced bone (femur metaphysis) weakening revealed through biomechanical analysis of Ultimate stress and Yield point. Ultimate stress (left panel), and Yield point (right panel) were measured by 3-point bending test in midshaft of femur from ovariectomized female mice as in FIGS. 2YA-B, administered with the respective treatment starting 2 months flowing the surgery. Test is performed postmortem, and mice are sacrificed on day 60 after staring the administration. (n=6-10 per group, data points show mean values+SD). Significance is calculated using Dunnet's multiple comparison One-way Anova test).

FIG. 10: Synergistic effect of Alendronate and HI P. goldsteinii in reverting ovariectomy-induced bone (femur metaphysis) weakening revealed through biomechanical analysis of Elastic energy and Stiffness. Elastic energy and Stiffness were measured by 3-point bending test in midshaft of femur from ovariectomized female mice as in FIGS. 2YA-B, administered with the respective treatment starting 2 months flowing the surgery. Test is performed postmortem, and mice are sacrificed on day 60 after staring the administration. (n=6-10 per group, data points show mean values+SD). Significance is calculated using Dunnet's multiple comparison One-way Anova test).

FIGS. 11A-B: Synergistic effect of Alendronate and P. goldsteinii (HI and Live form) on preventing ovariectomy-induced trabecular bone remodeling. Increased trabecular bone volume (FIG. 11A) and bone volume to total volume ratio (FIG. 11B) of femur in mice treated with either HI or Live Parabacteroides goldsteinii are shown.

FIGS. 12A-B: Synergistic effect of Alendronate and P. goldsteinii (HI and live form) on preventing ovariectomy-induced bone remodeling. Trabecular thickness (FIG. 12A) and cortical thickness of femur (FIG. 12B) are shown.

FIGS. 13A-B: Synergistic effect of alendronate and P. goldsteinii (HI and live form) on preventing ovariectomy-induced trabecular bone remodeling. Effects on ovariectomy-induced reduction trabecular number (FIG. 13A) and trabecular connectivity density (FIG. 13B) of femur in mice are shown.

FIGS. 14A-B: Synergistic effect of Alendronate and P. goldsteinii (HI and Live form) on preventing ovariectomy-induced trabecular bone remodeling. Results for trabecular spacing (FIG. 14A) and reduction in trabecular mineralization (FIG. 14B) are shown.

FIGS. 15A-D: Synergistic effect of Alendronate and HI P. goldsteinii on preventing ovariectomy-induced bone fragility. Results on Ultimate strength (FIG. 15A), Yield point (FIG. 15B), Elastic energy (FIG. 15C) and Stiffness (FIG. 15D) are shown.

FIGS. 16A-B: Supplementation of HI P. goldsteinii with alendronate decreases ovariectomy-induced body weight gain. Results for body weight relative to day 0 (FIG. 16A) and the corresponding area under the curve (FIG. 16B) are shown.

FIGS. 17A-B: Supplementation of HI P. goldsteinii with alendronate improves oral glucose tolerance in post-menopausal osteoporosis mice model. Results for glucose tolerance (FIG. 17A) and the corresponding area under the curve (FIG. 17B) are shown.

FIGS. 18A-B: Supplementation of HI P. goldsteinii increases secretion of Procollagen Type-1 N Terminal Propeptide (P1NP, bone formation marker), measured at Day 29 (FIG. 18A), or Day 50 (FIG. 18B) since the beginning of the treatment.

FIGS. 19A-F: Weights: Live vs. heat-inactivated direct comparison (obesity).

FIGS. 20A-D: Glycemia: Live vs. heat-inactivated direct comparison.

FIGS. 21A-D: Glycemia: Live vs. heat-inactivated direct comparison.

FIGS. 22A-B: Femur microCT cortical: Live vs. heat-inactivated direct comparison.

FIG. 23: Supplementation of HI P. goldsteinii increases the gene expression of osteoblast markers without affecting osteoclasts.

### DESCRIPTION OF ILLUSTRATIVE EMBODIMENTS

### I. Definitions

A “bacterial composition” is a composition that comprises one of more types of bacteria (e.g., live, dried, or heat-inactivated) or extracellular vesicles (i.e., secreted extracellular vesicles) from bacteria. In some embodiments, the bacteria are from the Clostridiaceae, Lactobacillaceae, and/or Porphyromonadaceae families. Specific bacteria that are contemplated include Lactobacillus gasseri, Lactobacillus reuteri, and Parabacteroides goldsteinii (e.g., live or inactivated P. goldsteinii). In some preferred embodiments, an inactivated Parabacteroides goldsteinii is used that has been inactivated using heat, freezing, or drying.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a bacterial composition means that amount of the bacterial composition which, when administered to a subject or patient for treating or preventing a disease, is an amount sufficient to affect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of anti-adherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “patient” or “subject” refers to a living mammalian organism, such as a human, monkey, cow, sheep, goat, dog, cat, mouse, rat, guinea pig, or transgenic non-human species thereof. In certain embodiments, the patient or subject is a primate. Non-limiting examples of human patients are adults, juveniles, and infants.

A “pharmaceutically acceptable carrier,” “drug carrier,” or simply “carrier” is a pharmaceutically acceptable substance formulated along with the active ingredient that is involved in carrying, delivering and/or transporting a biological agent. Carriers may be used to improve the delivery and the effectiveness of the active ingredient, including for example, controlled-release technology to modulate drug bioavailability, decrease drug metabolism, and/or reduce drug toxicity. Some carriers may increase the effectiveness of delivery of the active ingredient to the specific target sites. Examples of carriers include liposomes, microspheres (e.g., made of or comprising poly(lactic-co-glycolic) acid), albumin microspheres, synthetic polymers, nanofibers, protein-DNA complexes, protein conjugates, erythrocytes, virosomes, hydrogels, starches, and dendrimers. In some embodiments, the carrier comprises an enteric coating (e.g., a fatty acid, a wax, a shellac, a plastic such as a phthalate, CAP, CAT, PVAP, HPMCP, or a plant fiber) to reduce or slow degradation in the stomach, chitosan-alginate beads, or a hydrogel.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

### II. Bisphosphonates

Bisphosphonates are a class of drugs used to treat bone disorders. Bisphosphonates are anti-resorption drugs, which can promote bone density and can be taken orally or intravenously (Drake et al., 2008). Bisphosphonates can bind to free hydroxyapatite on the bone surface and resists enzyme digestion. In the process of bone resorption, bisphosphonate can be desorbed from hydroxyapatite and absorbed by osteoclasts, reducing the metabolic activity of osteoclasts, weakening the bone resorption capacity of osteoclasts, preventing osteoblasts and osteocytes from apoptosis, and increasing the number and function of osteoblasts (Zhang et al., 2022). Bisphosphonates can affect osteoclasts, protecting the bone formation function of mature osteoblasts and maintaining the osteocyte network. Bisphosphonates are widely used anti-resorption drugs for treating osteoporosis. It is anticipated that administration of an inactivated P. goldsteinii in combination with a bisphosphonate can reduce the dosage of the bisphosphonate needed to produce a therapeutic response, thus reducing or eliminating one or more side effect associated with administering higher dosages of the bisphosphonate.

A variety of bisphosphonates can be used in embodiments of the present disclosure. The bisphosphonate may be alendronate, risedronate, ibandronate, zoledronic acid, denosumab, raloxifene, or bazedoxifene.

In some embodiments, the bisphosphonate is alendronate (alendronic acid). Alendronate is also sold under the brand name FOSMAX. Alendronate has the structure:

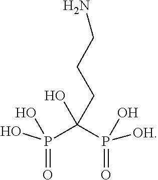 [SMILES: NCCCC(O)(P(=O)(O)O)P(=O)(O)O]

The alendronate may be in salt form, such as for example alendronate sodium or alendronate sodium trihydrate.

Alendronate can be administered in a variety of compositions and dosages. For example, alendronate can be administered intravenously or orally. In some embodiments, alendronate is administered to a mammalian subject such as a human subject in an amount of about 5, 10, 15, 20, 25, 30, 35, 40, 45, 50, 55, 60, 65, 70, 75, 80, 85, 90, 95, or 100 milligrams (mg), or any range derivable therein (e.g., once per day or once per week). The alendronate may be administered 1, 2, or 3 times a week or per month. In some embodiments, alendronate is administered to a human subject in an amount of about 60-75 mg (e.g., about 70 mg) or 25-50 mg (e.g., 35 mg) once per week. Other dosages that may be used to treat a mammalian subject such as a human include from about 1 mg to about 1 g, administered 1, 2, or 3 times per week or per month. In some embodiments, alendronate is administered to the subject once per week. In some embodiments, alendronate is included in nanoparticles, e.g., as described in Nguyen, 2016. Alendronate may be included in a pharmaceutical composition formulated for oral or intravenous administration. In some embodiments, the alendronate is administered to a human subject at a dose of about 1-20 mg daily, or about 1, 2, 3, 4, 5, 6, 7, 8, 9, 10, 11, 12, 13, 14, 15, 16, 17, 18, 19, or 20 mg per day, or any range derivable therein. For example, 5 mg or 10 mg per day may be administered to the subject per day. It is anticipated that a wide variety of salts of alendronate may be used such as, e.g., alendronate sodium. In some embodiments, the alendronate is administered at a dose or less than 5 mg per day or less than 35 per week or per month. In some embodiments, alendronate is administered intravenously, intraperitoneally, subcutaneously, or orally.

In some embodiments, administering less than 5 mg per day or less than 35 per week of alendronate in combination with an inactivated P. goldsteinii (e.g., heat-inactivated P. goldsteinii) can reduce or eliminate one or more side effects of alendronate that can result from dosages of 5-10 mg per day or 35-70 mg per week or higher. These side effects that are reduced or eliminated may include one or more of: pain (e.g., in the torso, jaw, joints, muscles, etc.), heartburn, bone pain, muscle pain, joint pain, stomach pain or nausea, diarrhea, and/or constipation.

In some embodiments, the bisphosphonate is risedronate (risedronic acid). The risedronate may be administered at a dose of 1, 2, 3, 4, 5, 6, 7, 8, 9, 10, 11, 12, 13, 14, 15, 16, 17, 18, 19, or 20 mg per day, or any range derivable therein. For example, 5 mg or 10 mg per day may be administered to the subject per day. The risedronate may be administered at a dose of 10, 15, 20, 25, 30, 35, 40, 45, 50, 55 mg per week. The risedronate may be administered at a dose of 125-175 mg (e.g., 150 mg) per month. It is anticipated that a wide variety of salts of risedronate may be used such as, e.g., risedronate sodium. The risedronate may be comprised in a pharmaceutical composition with an enteric coating.

### III. Inactivated Parabacteroides goldsteinii

Inactivated Parabacteroides goldsteinii can be produced via a variety of methods. In some embodiments, the inactivated Parabacteroides goldsteinii is inactivated via exposure to heat, for example as described in PCT/US20/51581 and PCT/US19/53402. It is anticipated that other methods of inactivation can be used to generate inactivated Parabacteroides goldsteinii that may exert similar effect(s) and can be used for the treatment of a metabolic disease or disorder as described herein (e.g., obesity, diabetes, etc.). For example, the inactivated P. goldsteinii can be generated via exposure of the bacteria to light (e.g., radiation or ionizing radiation), air plasma, pressure (e.g., ultrasound under pressure, high hydrostatic pressure), a peroxide (e.g., hydrogen peroxide), an alcohol (e.g., ethanol), exposure to cold temperatures or freezing, dehydration, lyophilization, or pulsed electric field (PEF); in some embodiments, one or more of the foregoing methods for inactivation of bacteria can be used in combination with application of heat in order to produce inactivated P. goldsteinii that can be used, e.g., for the treatment of a metabolic disease or disorder as described herein. In some embodiments, a composition as described herein may include both live and inactivated Parabacteroides goldsteinii.

A variety of methods can be used to generate heat-inactivated Parabacteroides goldsteinii. For example, the bacteria may be heated to at least 95° C., at least 100° C., or about 100° C. for at least 10 minutes, 10-20 minutes, or about 15 minutes, e.g., as described in Wu et al. (2019). It is anticipated that autoclaving or heating in a solution (e.g., boiling in water) can also be used. Generally, heat may inactivate the bacteria via one or more of: membrane damage, loss of nutrients and ions, ribosome aggregation, DNA strand breaks, inactivation of essential enzymes, and protein coagulation. Additional methods of inactivation that may be used with the present invention are described, e.g., in Lin et al. (2015). After generating the inactivated (e.g., heat-inactivated) P. goldsteinii, the bacteria can be subsequently dried, frozen, or lyophilized, if desired. In some preferred embodiments, Parabacteroides goldsteinii are killed using heat inactivation.

Inactivated P. goldsteinii can also be generated based on exposure to radiation, such as ionizing radiation. In some embodiments, the bacteria can be inactivated by exposure to light comprising or consisting of light having a wavelength of about 405 nm. For example, a light-emitting diode (LED) array producing light with a wavelength of about 405 can be used to inactivate bacteria (e.g., Maclean et al., 2009). In some embodiments, the radiation may be ultraviolet (UV) radiation having a wavelength of from about 240 nm to about 280 nm. In some embodiments, the radiation is ionizing radiation, such as x-rays.

The inactivated Parabacteroides goldsteinii can be generated via exposure to a peroxide, such as for example hydrogen peroxide. The hydrogen peroxide may be contacted with the bacteria in a solution. In some embodiments, the bacteria are contacted with a hydrogen peroxide vapor in order to inactivate the bacteria. For example, the bacteria may be exposed to hydrogen peroxide vapor of about 10-100 mg/m³ (ppm) for about 1.5-48 hours (e.g., Malik et al., 2013). Hydrogen peroxide can be applied either as a liquid or as a vapor for inactivating bacteria. In some instances, the mode of action of hydrogen peroxide in vapor form may result in increased intensive oxidation of a range of biological macromolecules than do aqueous solutions of hydrogen peroxide (Finnegan et al., 2010). Similar to heat-inactivation, using hydrogen peroxide for inactivation of bacteria has the advantage that it decomposes into non-toxic byproducts after reacting with a bacterium. In some embodiments, it is anticipated that a solution of 1-2% hydrogen peroxide can be contacted with P. goldsteinii for about 5-10 minutes in order to inactivate the bacteria. In some embodiments, a combination of heat and hydrogen peroxide can be used to inactivate P. goldsteinii.

Other methods for inactivating P. goldsteinii can be used. For example, the bacteria may be inactivated by contacting the bacteria with an alcohol (e.g., ethanol, methanol, propanol, or isopropanol) at a particular concentration (e.g., at least 70% v/v alcohol, such as 70% ethyl alcohol) in order to inactivate the bacteria.

The inactivated Parabacteroides goldsteinii may also be generated via exposure to air plasma, such as a direct-current, cold-atmospheric-pressure air plasma microjet (e.g., Tian et al., 2010); for example, after about 10 min of plasma treatment, a decrease in the pH may be observed due to the reaction of NOx produced in the air plasma with water at the gas-liquid interface.

The inactivated Parabacteroides goldsteinii can be generated via exposure to ionizing irradiation (e.g., gamma rays produced from cobalt-60, electron beams, or X-rays). Generally, the dosage of the ionizing radiation applied to the bacteria is preferably sufficient to damage DNA of the bacteria and/or prevent further growth of the bacteria.

Ultrasound under pressure or high hydrostatic pressure (HHP) can also be used to inactivate bacteria. In some embodiments, pressures of from about 100 to 1000 MPa are applied in order to inactivate bacteria. The effectiveness of HHP has been demonstrated in the field of food sanitization. Ultrasound is defined as sound waves with frequencies above the threshold for human hearing (>16 kHz). In some embodiments, ultrasound application can be used in combination with an external hydrostatic pressure (e.g., up to 600 kPa of manosonication (MS)) and/or application of heat in order to inactivate the P. goldsteinii.

Pulsed electric field (PEF) can also be (e.g., Manas et al., 2005). PEF approaches generally involve the application of short duration (e.g., 1-100 μs) high electric field pulses (10-50 kV cm⁻¹) to sample between two electrodes. In various embodiments, it is anticipated that a combination of one or more of the foregoing methods may be used to produce the inactivated P. goldsteinii.

It is anticipated that a variety of amounts of inactivated (e.g., heat inactivated) P. goldsteinii can be administered to a mammalian subject. For example, in mice, 200 μl bacteria with concentration of about 1,5-2.5 OD/ml at 600 nm corresponding to 2.4-4*10⁸ cells (200 μl of 1.5-2.5 OD/ml), equivalent to about 1.2-2*10¹⁰ cells/kg mouse BW, were administered every other day. For humans, equivalent dose is about 0.97-1.62*10⁹ cells/kg BW, or 6.8-11.3*10¹⁰ cells for a 70 kg subject every other day (alternatively per day); or alternative proposed range of 10⁹-10¹⁰ per day (Depommier et al., 2019), or ranges between 10⁸-10¹³ cells per day or every other day or 10⁸-10¹⁵ per week or per month can be administered.

### IV. Spermine and Spermidine

In some aspects, spermine and/or spermidine may be enterically administered to a mammalian subject, such as a human, to treat a metabolic disease or disorder as described herein (e.g., diabetes, obesity, etc.). Spermidine (N-(3-(aminopropyl)-1,4-butane diamine) and spermine (N, N′-bis (3-aminopropyl)-1,4-butane diamine) are naturally occurring polyamines, and can function as regulators for a variety of cellular processes including DNA stability, transcription, translation, apoptosis, and may affect cellular growth and differentiation (Igarashi et al., 2010). In some studies, spermine and spermidine inhibited experimental inflammation in association with suppressed expression of pro-inflammatory cytokines (Soda et al., 2005). Spermine and spermidine may affect osteoclast differentiation (Yamamoto et al., 2012), and a correlation between polyamine levels and symptoms of skeletal muscle hypertrophy (Turchanowa et al., 2000), Alzheimer's disease (Morrison et al., 1995), and ischemia (Paschen et al., 1987) have been observed.

A variety of dosages of spermine and/or spermidine may be enterically administered to the subject, preferably a human to treat a metabolic disease or disorder as described herein. Spermine and/or spermidine can be administered in concentrations of from about 0.3 mM to about 3 mM each, or more preferably about 0.3-1 mM, or about 0.5 mM (e.g., orally administered, for example in drinking water), and these concentrations were shown to be effective in mouse models of osteoporosis and aging. In experimental studies 0.5 mM in drinking water, which is the equivalent of 18.2 mg/kg body weight, also showed beneficial effects of polyamine supplementation on ovariectomy induced bone loss and lifespan extension. In some embodiments, a range between 1-50, 2-40, 5-25, or 15-20 mg/kg body weight per day, or any range derivable therein, may be used to treat a metabolic disease or disorder as described herein.

In some embodiments, microbiota is cultured in spermine and/or spermidine prior to administration to a mammalian subject, such as a human, to treat a metabolic disease or disorder. For example, the microbiota can be cultured in 0.1-10 mM, 1-7 mM, or more preferably 1-5 mM, or 0.5, 1, 2, 3, 4, or 5 mM, or any range derivable therein, of spermine and/or spermidine, prior to administration to the subject to treat the metabolic disease or disorder. The microbiota may comprise Parabacteroides goldsteinii, Lactobacillus reuteri and/or Lactobacillus gaseri. In some embodiments, the Parabacteroides goldsteinii that has been cultured or expanded in spermine and/or spermidine is subsequently inactivated via a method as described herein (e.g., heat inactivation, exposure to hydrogen peroxide, etc.), and the inactivated P. goldsteinii can be included in a pharmaceutical or probiotic composition or enterically administered to a human to treat a metabolic disease or disorder as described herein. In some embodiments, the microbiota includes living and/or inactivated Akkermansia mucuniphila in combination with Parabacteroides goldsteinii (living and/or inactivated), Lactobacillus reuteri and/or Lactobacillus gaseri; and in some embodiments, the microbiota includes both living and inactivated Akkermansia mucuniphila.

### V. Additional Bacteria

In some embodiments, a combination therapy comprising P. goldsteinii (e.g., inactivated P. goldsteinii) and a bidphosphate (e.g., alendronate) are administered to a mammalian subject further in combination with an additional bacteria. In some embodiments, the additional bacteria is a Lactobacillus spp. (e.g., Lactobacillus gasseri or Lactobacillus reuteri), Bifidobacteriaceae spp. (e.g., Bifidobacterium longum), Akkermansia spp. (e.g., Akkermansia muciniphila), or other Parabacteroides species (e.g., P. johnsonii and other). The Lactobacillus may comprise Lactobacillus reuteri, Lactobacillus acidophilus, and/or Lactobacillus rhamnosus or other Lactobacillus species. In some preferred embodiments, the additional bacteria are alive.

In various embodiments, it is anticipated that 1, 2, 3, 4, 5, 6, or more of the following types of bacteria may be administered to the subject to treat a metabolic disease or may be included in a pharmaceutical composition or probiotic composition disclosed herein. For example, the 1, 2, 3, 4, 5, 6, or more of Clostridialeace-assimilate spp., Lactobacillus spp. (e.g., Lactobacillus reuteri, Lactobacillus gasseri, Lactobacillus acidophilus, and/or Lactobacillus rhamnosus), Bifidobacteriaceae spp. (e.g., Bifidobacterium longum), Parabacteroides spp. (e.g., Parabacteroides goldsteinii) and Akkermansia spp. (e.g., Akkermansia muciniphila) may be included in a pharmaceutical or probiotic composition disclosed herein and/or administered to a mammalian subject, such as a human patient, to treat a metabolic disease or disorder. Lactobacillus species such as Lactobacillus reuteri (Britton et al., 2014; also recently described in humans in Nilsson et al., 2018), Lactobacillus acidophilus (Dar et al., 2018), and/or Lactobacillus rhamnosus (Li et al., 2016) may be included in compositions for the treatment of a metabolic disease or disorder.

### VI. Extracellular Vesicles of Bacteria

In some embodiments, extracellular vesicles from P. goldsteinii may be administered to a subject to treat a metabolic disease or disorder. For example, extracellular vesicles (EVs) may be produced via methods as described, for example, in Chelakkot et al., 2018, or Choi et al., 2015. It is anticipated that EVs from Parabacteroides goldsteinii, Lactobacillus gasseri, or Lactobacillus reuteri may be used to treat a metabolic disease or disorder as described herein (e.g., obesity, type 2 diabetes, fatty liver).

Extracellular vesicles (EVs) are lipid bilayer structures secreted from the gut microbiota, including from both Gram-negative and -positive bacteria (Ellis and Kuehn, 2010 and Lee et al., 2009). A variety of bacteria constitutively produce EVs, defined as spherical lipid bilayers with an average diameter of 20-200 nm (Lee et al., 2007). EVs are composed of proteins, lipids, nucleic acids, lipopolysaccharides, and other virulence factors associated with pathogenesis (Horstman and Kuehn, 2002, Hong et al., 2011, and Kim et al., 2013). EVs released by bacteria may have diverse roles in the microbial community, and some data suggests that they may transfer genetic material and proteins from the bacteria to the host (Kuehn and Nesty, 2005). EVs may directly interact with immune cells and epithelial cells to initiate several signaling pathways and may affect or mediate host-pathogen interactions.

In some embodiments, EVs may be prepared via the following approach. Bacterial species or warm microbiota may be cultured under aerobic or anaerobic conditions (e.g., 95% N₂ 5% CO₂ at 37° C.) until desired (e.g., when the optical density at 600 nm reaches 1.5, as previously described; Derrien et al., 2004). Isolation of EVs may be performed as previously described in Kang et al., 2013. More specifically, bacterial cultures may be pelleted at 10 000 g for 20 min, and the supernatant may then be filtered through a 0.45-μm vacuum filter. The filtrate can be enriched, e.g., using QuixStand (GE Healthcare, Little Chalfont, UK) and subsequently filtered through a 0.22-μm bottle-top filter. The filtrate may then be pelleted by ultracentrifugation (e.g., in a 45 Ti rotor at 150 000 g for 2 h at 4° C.). The final pellets may then be resuspended in phosphate-buffered saline (PBS) and stored at −80° C. EVs may be analyzed, if desired, by transmission electron microscopy, dynamic light scattering, and/or sodium dodecyl sulfate-polyacrylamide gel electrophoresis (SDS-PAGE) followed by gel staining with Coomassie Brilliant Blue R-250. The amount of protein or DNA extracted from the EVs can be measured and used to evaluate the quantity of EVs obtained.

### VII. Pharmaceutical Formulations and Routes of Administration

P. goldsteinii (e.g., live or inactivated, such as heat-inactivated P. goldsteinii) and a bisphosphonate (e.g., alendronate), can be included in the same or separate pharmaceutical or probiotic compositions for administration to a mammalian subject. The pharmaceutical composition may optionally comprise additional bacteria as described herein, spermine, spermidine, an anti-obesity drug, or a drug for treating type II diabetes.

Pharmaceutical formulations (also referred to as bacterial formulations or pharmaceutical compositions) comprising a therapeutically effective amount of a live or inactivated bacterial composition disclosed herein can be formulated with one or more excipients and/or carriers appropriate to the indicated route of administration (e.g., delivery to the gastrointestinal tract). In some embodiments, the bacteria disclosed herein are formulated in a manner amenable for the treatment of human and/or veterinary patients. In some embodiments, formulation comprises admixing or combining one or more of the bacteria disclosed herein (e.g., warm microbiota, and/or heat-inactivated Parabacteroides goldsteinii) with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the bacteria may be slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain carriers and/or excipients such as preservatives, stabilizers, wetting agents, emulsifiers, encapsulating agents such as lipids, dendrimers, polymers, proteins such as albumin, nucleic acids, and buffers. In some embodiments, the pharmaceutical composition comprises both an inactivated or live P. goldsteinii and alendronate.

In some embodiments, the pharmaceutical formulation comprises inactivated (e.g., heat-inactivated) Parabacteroides goldsteinii. A variety of amounts of inactivated Parabacteroides goldsteinii may be included, for example from about 1×10⁸ to about 1×10¹³ cfu. In some embodiments, the inactivated Parabacteroides goldsteinii is included in a pharmaceutical composition or a probiotic composition formulated for oral or enteric administration. For single, or mixed (cultivated) bacteria administration, the inactivated Parabacteroides goldsteinii can be administered orally (e.g., in the form of tablets). For the fecal microbiota transplantation from donors (FMT), heat-inactivated Parabacteroides goldsteinii can for example be added to the microbiota that is delivered to the gastrointestinal system, e.g., via nasogastric tube or intracolonically.

Bacterial formulations may be administered by a variety of methods, e.g., orally, intracolonically, intranasally, intrarectally, via a catheter, via a lavage, via a nasogastric tube, via local delivery, or via a method for fecal microbiota transplantation (FMT). Depending on the route of administration, the bacterial compositions disclosed herein may be coated in a material to protect the bacterial compositions from the action of acids and other natural conditions which may inactivate the bacterial compositions. To administer the bacterial composition, it may be necessary to coat the bacterial composition with, or co-administer the bacterial composition with, a material to prevent its inactivation. In some embodiments, the bacterial composition may be administered to a patient in an appropriate carrier, for example, polymers, hydrogels, liposomes, starches, or a diluent. Pharmaceutically acceptable diluents include saline and aqueous buffer solutions. Liposomes include water-in-oil-in-water CGF emulsions as well as conventional liposomes.

Formulations may be employed to protect the bacterial compositions from the harsh gastric environment (Govander et al., 2014). Gastro-resistant polymers and coatings have been shown to supply protection against the harsh gastric environment. These coatings included enteric coated tablets and capsules that site-specifically deliver the administered probiotic bacteria to the intestinal system. These enteric coats are often pH selective and allow for protection against the harsh gastric conditions and subsequently dissolve in the alkali media of the intestinal system (Calinescu et al., 2005 and Yang et al., 2002). Non-limiting examples of excipients that may employed in the formulation of bacterial compositions are hydroxypropyl methylcellulose phthalate and carboxymethyl high amylose starch. Excipients may be combined to enhance delivery of the bacterial composition to the gastrointestinal tract. For example, carboxymethyl high amylose starch may be combined with chitosan for delivery of the bacterial composition to the colon. Formulations may include different polymers with different properties, or similar polymers with different properties, depending on the site of intended delivery to deliver the bacterial composition to different areas of the gastrointestinal tract (Yang et al., 2002).

The bacterial compositions disclosed herein may also be administered orally, intracolonically, intranasally, intrarectally, via a catheter, via a lavage, via a nasogastric tube, via local delivery, or via a method for fecal microbiota transplantation (FMT). The bacterial composition may be in the form of a dispersion. Dispersions can be prepared in glycerol, liquid polyethylene glycols, and mixtures thereof and in oils.

In some embodiments, the carrier comprises an enteric coating to reduce or slow degradation in the stomach. For example, the enteric coating may be a fatty acid, a wax, a shellac, a plastic such as a phthalate, CAP, CAT, PVAP, HPMCP, or a plant fiber (e.g., Hussan et al., 2012). In some embodiments, the pharmaceutical or probiotic composition may contain chitosan-alginate beads, or a hydrogel. Nonetheless, it is anticipated that in some embodiments,

The bacterial compositions disclosed herein can be administered orally, for example, with an inert diluent or an assimilable edible carrier. The bacterial compositions and other ingredients may also be enclosed in a hard or soft-shell gelatin capsule, compressed into tablets, or incorporated directly into the patient's diet. For oral therapeutic administration, the bacterial compositions disclosed herein may be incorporated with excipients and used in the form of ingestible tablets, buccal tablets, troches, capsules, elixirs, suspensions, syrups, wafers, and the like. The percentage of the therapeutic agent in the compositions and preparations may, of course, be varied. The amount of the therapeutic agent in such pharmaceutical formulations is such that a suitable dosage will be obtained.

In some embodiments, the effective dose range for the therapeutic agent can be extrapolated from effective doses determined in animal studies for a variety of different animals. Precise amounts of the therapeutic composition depend on the judgment of the practitioner and are specific to each individual. Other factors affecting the dose include the physical and clinical state of the patient, the route of administration, the intended goal of treatment and the potency, stability, and toxicity of the particular therapeutic formulation.

The actual dosage amount of a bacterial composition of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

An inactivated P. goldsteinii and a bisphosphonate (e.g., alendronate) can be administered to a mammalian subject at the same time or at different times. Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The composition comprising a bacterial composition (e.g., an inactivated Parabacteroides goldsteinii) may be administered on a routine schedule. As used herein, a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

In some embodiments, the bisphosphonate (e.g., alendronate) is administered to the mammalian subject orally or intravenously in an amount of about 30-100, 50-80, 30, 40, 50, 60, 70, 80, 90, or 100 milligrams (mg), or any range derivable therein. The bisphosphonate may be included in separate or the same pharmaceutical formulation as a bacterial composition disclosed herein, such as a live or inactivated P. goldsteinii, for administration to the mammalian subject.

### VIII. Metabolic Diseases and Disorders

It is anticipated that a variety of metabolic diseases or disorders may be treated with the methods and bacterial compositions described herein. For example, a bacterial composition as described herein (e.g., live bacteria, heat inactivated bacteria, lyophilized bacteria, bacteria in a pharmaceutical composition, or secreted extracellular vesicles of the bacteria) may be administered enterically or to the gastrointestinal tract of a subject in combination with a bisphosphonate (e.g., alendronate) to treat a metabolic disease or disorder. In some embodiments, the bacterial composition is inactivated Parabacteroides goldsteinii. In some embodiments, the metabolic disease or disorder is obesity, fatty liver disease, type 2 diabetes, insulin intolerance, or dyslipidemia. In some embodiments, the metabolic disease results from menopause, such as pos-menopausal weight gain.

The prevalence of obesity, measured by body mass index, has risen to unacceptable levels in both men and women in the United States and worldwide with resultant hazardous health implications. Genetic, environmental, and behavioral factors influence the development of obesity. In adults, classification systems (World Health Organ Tech Rep Ser., 2000) and obesity guidelines (Jensen et al., 2014) define healthy body weight as a BMI between 18.5 and 24.9 kg/m², overweight between 25.0 and 29.9 kg/m², and obesity >30 kg/m². In children and adolescents, the U.S. Centers for Disease Control and Prevention (CDC) BMI-for-age growth charts define overweight as a BMI at or above the 90th percentile of standard weight and obesity as a BMI above the 95^{th} percentile of standard weight. Obesity is associated with and contributes to a shortened life span, type 2 diabetes mellitus, cardiovascular disease, some cancers, kidney disease, obstructive sleep apnea, gout, osteoarthritis, and hepatobiliary disease, among others (Bray et al., 2018). Weight loss can reduce these diseases in a dose-related manner, with the more weight lost, the better the outcome.

Fatty Liver Disease, also known as hepatic steatosis, is a condition where excess fat builds up in the liver. The fatty liver disease can be non-alcoholic fatty liver disease (NAFLD) or alcoholic liver disease. Non-alcoholic fatty liver disease (NAFLD) is a common cause of chronic liver disease, and its worldwide prevalence continues to increase with the growing obesity epidemic. Non-alcoholic fatty liver disease is the most common cause of elevated liver enzymes. Within the NAFLD spectrum, typically only non-alcoholic steatohepatitis progresses to cirrhosis and hepatocellular carcinoma (Vernon et al., 2011). With the growing epidemic of obesity, the prevalence and impact of NAFLD continues to increase.

Type 2 diabetes (T2D), formerly known as adult-onset diabetes, is a form of diabetes that is characterized by high blood sugar, insulin resistance, and relative lack of insulin. Type 2 diabetes can be diagnosed using a glycated hemoglobin (A1C) test to determine average blood sugar levels, and diagnosis can also be performed using a random blood sugar test, a fasting blood sugar test, or an oral glucose tolerance test. Type 2 diabetes is the most common form of diabetes and can be caused by several factors, including obesity, physical inactivity, and genes. Insulin can help achieve ideal hemoglobin A1c goals for patients with type 2 diabetes (Wallia et al., 2014). Obesity in some cases can result in insulin resistance and is common in people with type 2 diabetes. In some embodiments, a bacterial composition as disclosed herein (e.g., heat-inactivated Parabacteroides goldsteinii) is administered to a mammalian subject, such as a human, in combination with another therapy for type 2 diabetes such as, e.g., metformin, a sulfonylurea, a meglitinide, a thiazolidinedione, a DPP-4 inhibitor, a GLP-1 receptor agonist (e.g., exenatide), a SGLT2 inhibitor, or insulin.

Dyslipidemia is characterized by abnormal levels of lipids in the blood, such as: elevation of plasma cholesterol, triglycerides (TGs), or both; increased low-density lipoprotein (LDL) or very-low-density lipoprotein (VLDL) levels; low high-density lipoprotein cholesterol (HDL) levels; or low HDL cholesterol level. In some embodiments, the dyslipidemia is a hyperlipidemia (increase in blood lipids). Dyslipidemia may contribute to the development of atherosclerosis.

### IX. Bone Diseases

It is anticipated that a variety of bone diseases or disorders may be treated with the methods and bacterial compositions described herein. For example, a bacterial composition as described herein (e.g., live bacteria, heat inactivated bacteria, lyophilized bacteria, bacteria in a pharmaceutical composition, or secreted extracellular vesicles of the bacteria) may be administered enterically or to the gastrointestinal tract of a subject to treat a bone disease or disorder or promote or increase bone density. In some embodiments, the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, or Paget's disease. Without wishing to be bound by any theory, it is anticipated that the methods and bacterial compositions provided herein may be used to reduce cortical and/or trabecular bone loss, reduce cortical and/or trabecular bone mineral content loss, improve the bone biomechanical resistance, increase bone formation, and/or reduce bone-resorption.

In some embodiments, the disease is osteoporosis. Osteoporosis is the most prevalent of metabolic bone disorders, characterized by low bone mass and microarchitectural deterioration (Sozen et al., 2017). Patients with osteoporosis have fragile bones and are vulnerable to fractures. The most common type of primary osteoporosis is due to the post-menopausal estrogen deficiency, reflected in a higher incidence of osteoporosis in women (Reginster and Burlet, 2006).

### X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Live and Heat-Inactivated P. goldsteinii and Alendronate Supplementation in Curative In Vivo Model of Post-Menopausal Physiology

16-week old ovariectomized female C57BL/6J mice were used as an in vivo model of osteoporosis for experiments. After ovariectomy, increased body weight gain and blood glucose were observed. Subcutaneous Alendronate injection (80 μg/kg/week) and/or heat-inactivated P. goldsteinii were administered to mice. The following experimental groups were tested: (1) Sham mice gavaged with anaerobic PBS, (2) Ovariectomized mice with anaerobic PBS, (3), Ovariectomized mice with Alendronate (ALN) s.c injection, (4) Ovariectomized mice with 1.5 OD/ml Heat-inactivated (HI) Parabacteroides goldsteinii, (5) Ovariectomized mice with both heat-inactivated P. goldsteinii supplementation (n=8 to 10 mice per group). Mice were then tested for changes in body weight.

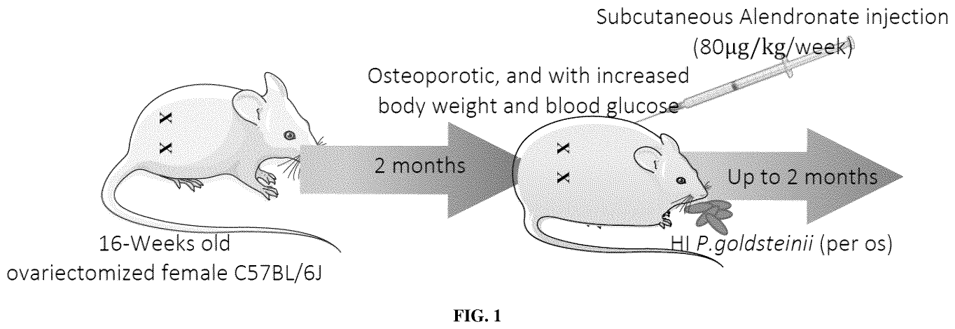

A curative mouse model for osteoporosis was used for experiments, as follows. 16-week old female C57Bl/6J mice were ovariectomized and left for 2 months to develop the symptoms of osteoporosis and postmenopausal weigh gain and glucose intolerance. Administration of the respective treatment was initiated following these 2 months after the ovariectomies, i.e, once the mice already developed the symptoms of the respective metabolic disease. An overview of this mouse model of osteoporosis is shown in FIG. 1.

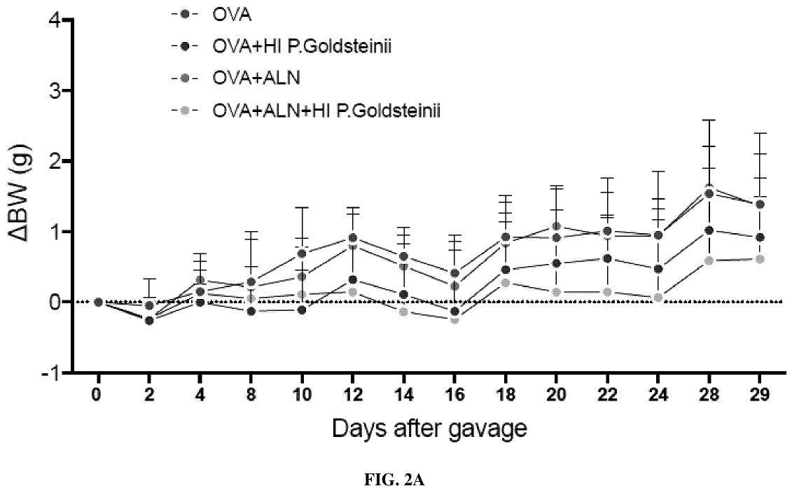

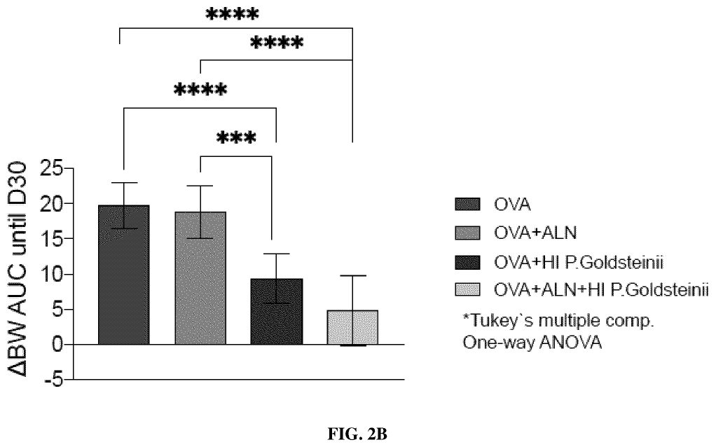

Body weight gain of ovariectomized female mice was measured starting 2 months after the surgery to address the curative effects of the treatment until day 29 after starting the administration. Mice were administered with the respective treatment starting at day 0, which is 2 months flowing the surgery. Results are shown in FIGS. 2A-B.

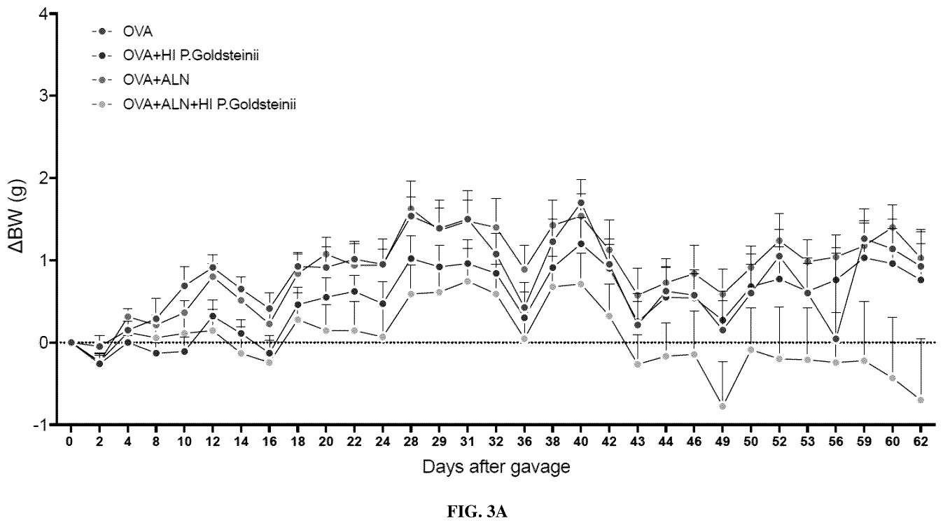

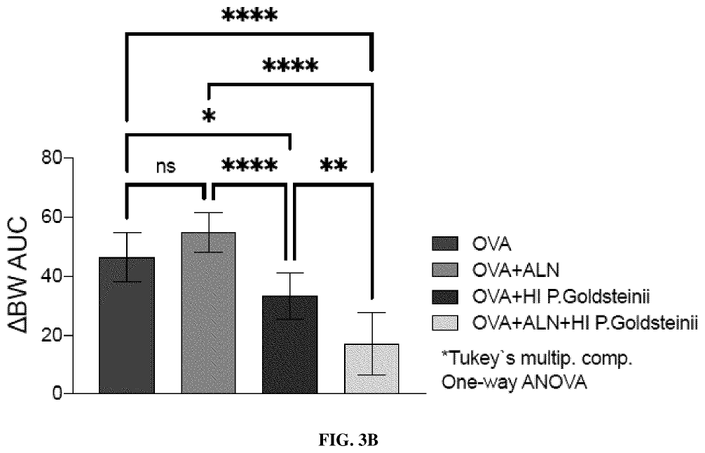

Body weight gain of ovariectomized female mice was measured starting 2 months after the surgery to address the curative effects of the treatment until day 60 after starting the administration. Mice were administered with the respective treatment starting at day 0, which is 2 months flowing the surgery. The values are normalized to the initial body weight at the start of the treatment. Results are shown in FIGS. 3A-B.

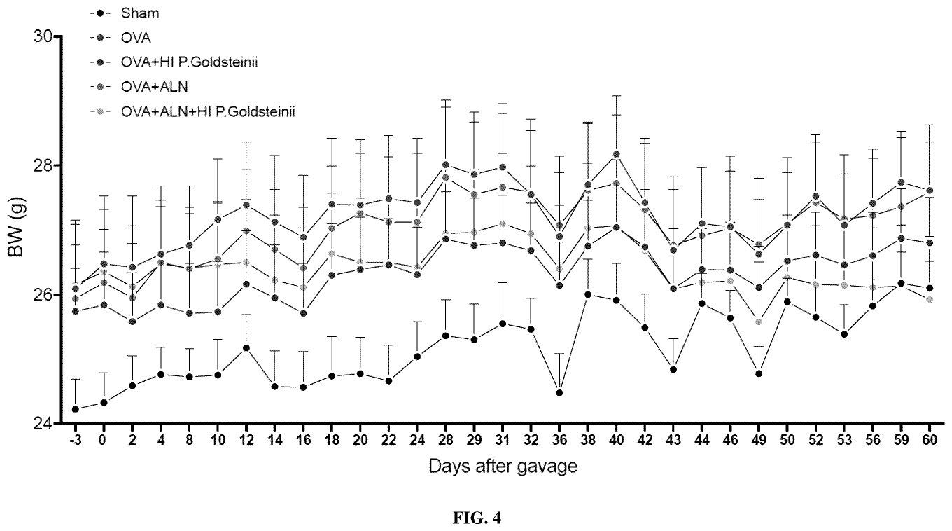

Absolute body weights of ovariectomized female mice, starting 2 months after the surgery to address the curative effects of the treatment until day 60, after starting the administration. Mice were administered with the respective treatment starting at day 0, which is 2 months flowing the surgery. Results are shown in FIGS. 4A-B.

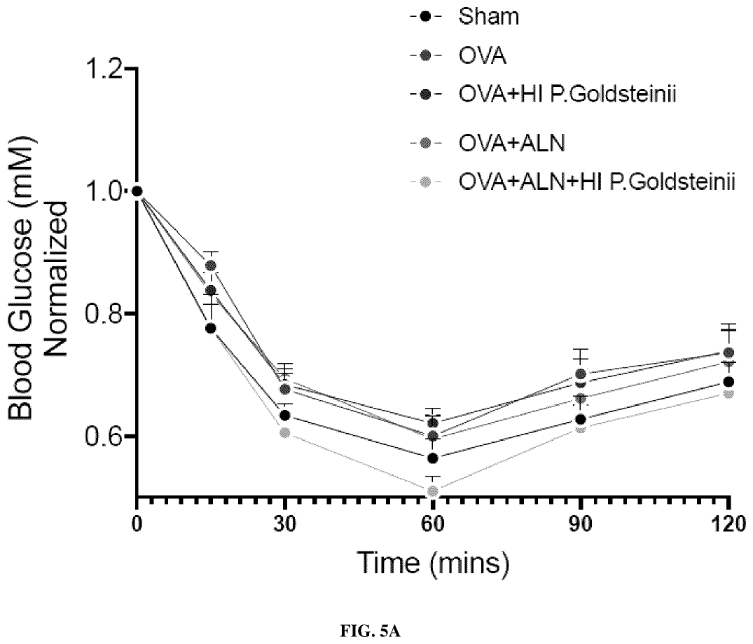

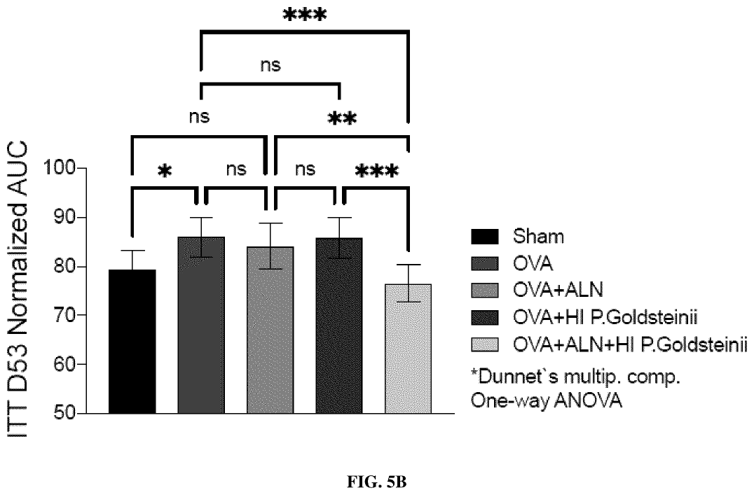

Insulin sensitivity (normalized values) was measured by intraperitoneal insulin tolerance test of ovariectomized female mice, administered with the respective treatment starting 2 months flowing the surgery, considered as day 0. Test is performed on day 52 after staring the administration. Results are shown in FIGS. 5A-B.

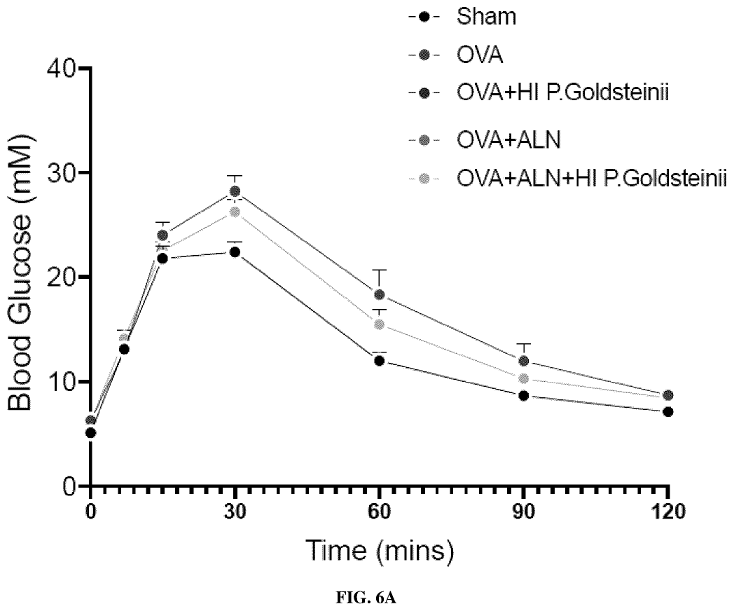

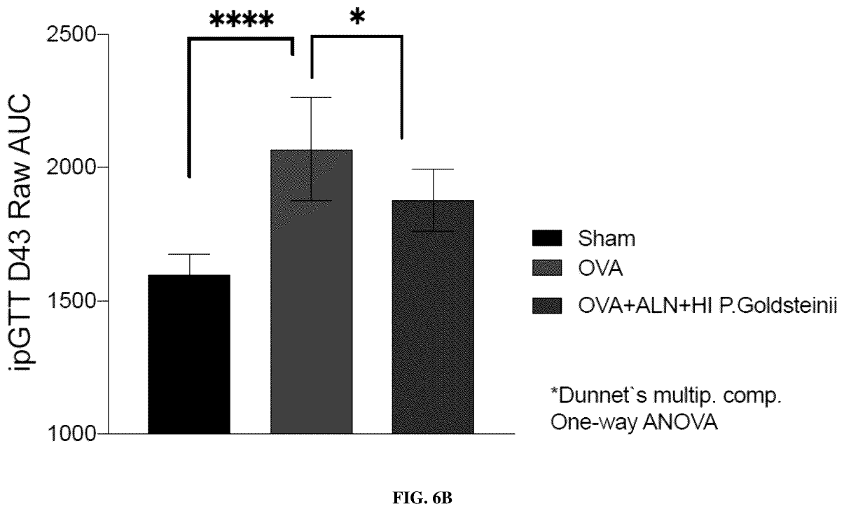

Glucose tolerance was measured by intraperitoneal glucose tolerance test of ovariectomized female mice, administered with the respective treatment starting 2 months flowing the surgery, considered as day 0. Test is performed on day 52 after staring the administration. Results are shown in FIGS. 6A-B.

Administering alendronate alone did not alter the body weight of mice. Supplementation of heat-inactivated P. goldsteinii with alendronate further decreased body weight gain in curative model of osteoporosis, as compared to administration of heat-inactivated P. goldsteinii alone.

Administration of both heat-inactivated (HI) P. goldsteinii and alendronate was also observed to improve glucose tolerance (ipGTT) in vivo.

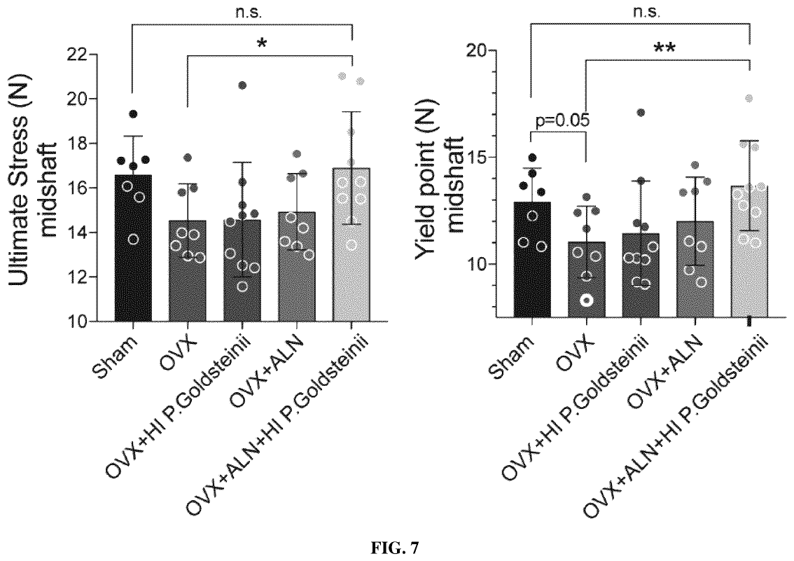

Alendronate and HI P. goldsteinii effects on reverting ovariectomy induced effects on bone in vivo were also tested. Although individually Alendronate and HI P. goldsteinii were not observed to result in improvements in bone properties based on ultimate stress and yield point tests in femur midshafts, when (HI) P. goldsteinii and alendronate were co administered, statistically significant improvements in in bone properties based on ultimate stress and yield point tests in femur midshafts were observed. Synergistic improvements from the combination were observed. Results are shown in FIG. 7.

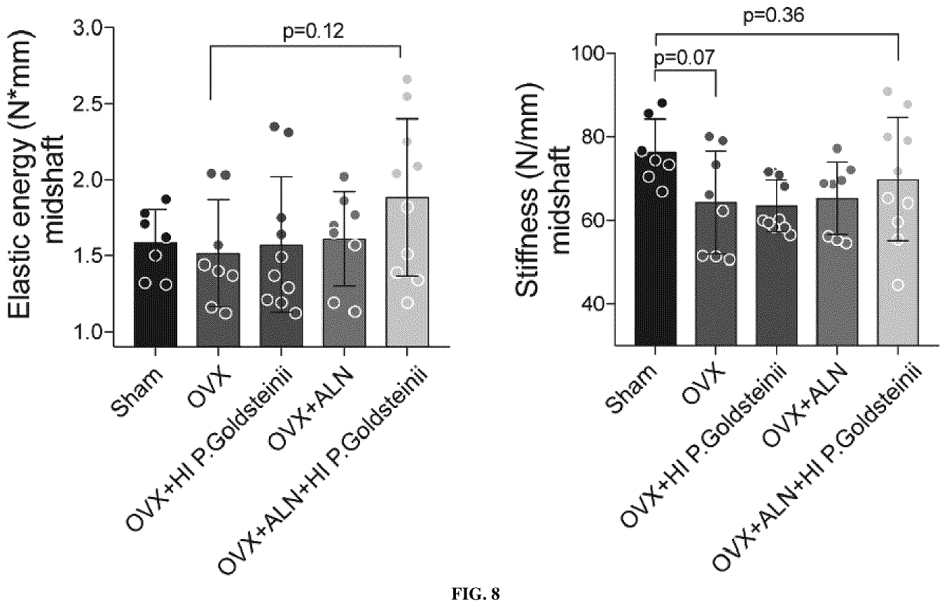

Additional bone properties were tested. No changes in Elastic energy and Stiffness were observed in femur midshaft after administration of alendronate and/or HI P. goldsteinii. Results are shown in FIG. 8.

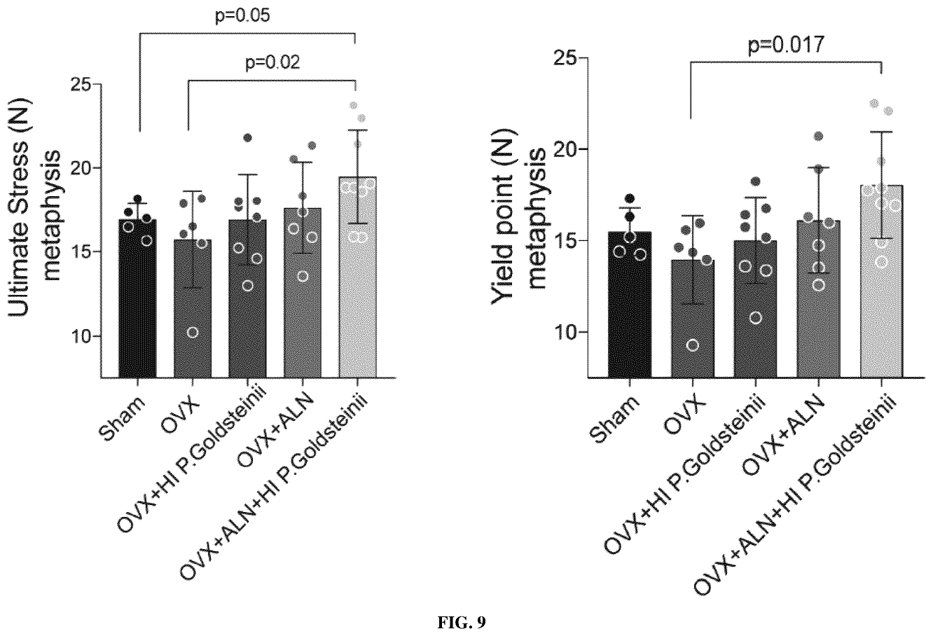

Alendronate and HI P. goldsteinii, alone or in combination, were tested administered to ovariectomized mice and ultimate stress and yield point parameters of femur metaphysis were tested. When administered individually, Alendronate and HI P. goldsteinii were not observed to result in a statistically significant increase in ultimate stress and yield point parameters of femur metaphysis. However, when administered in combination, alendronate and HI P. goldsteinii reverted ovariectomy induced decreases in ultimate stress and yield point in femur metaphysis. Surprisingly, this improvement resulted in ultimate stress and yield point in femur metaphysis that exceeded even the control mice. Synergistic effects of Alendronate and HI P. goldsteinii in reverting ovariectomy-induced bone (femur metaphysis) weakening were observed in biomechanical analysis of Ultimate stress and Yield point. Results are shown in FIG. 9.

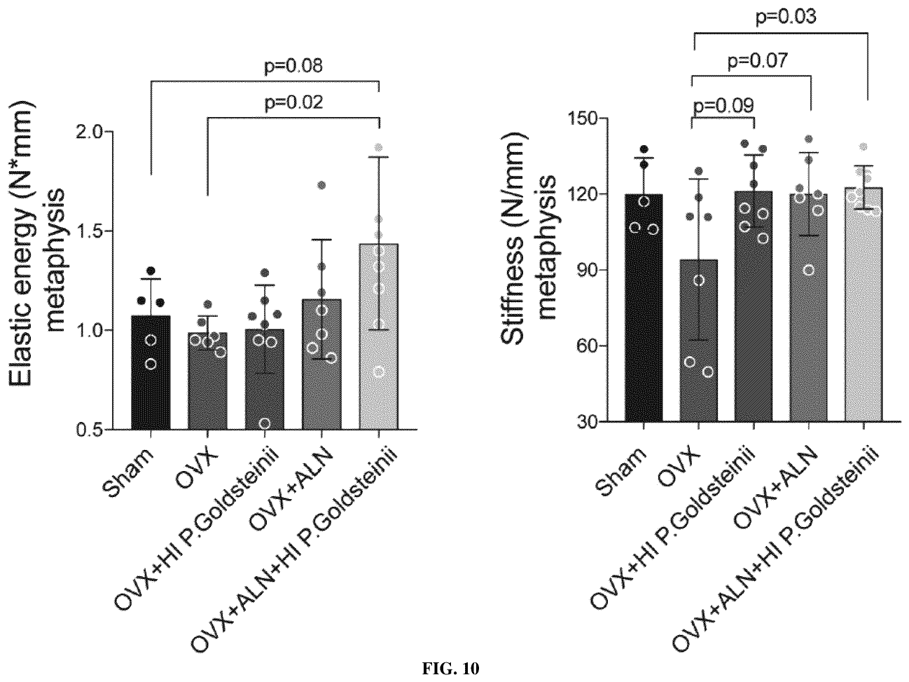

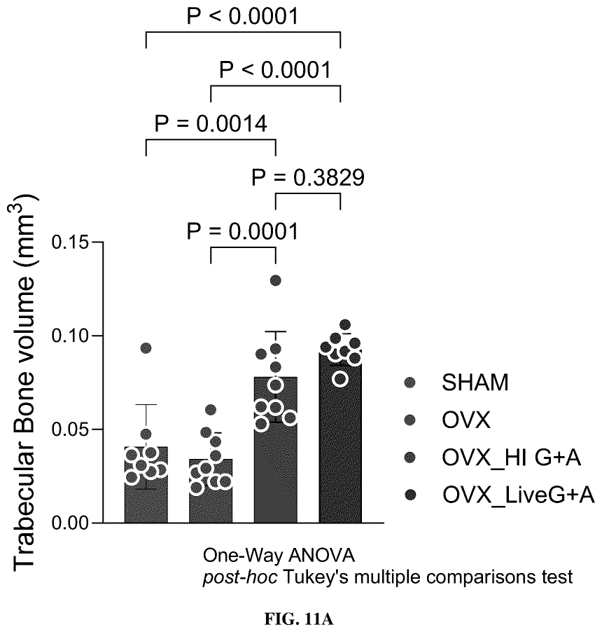

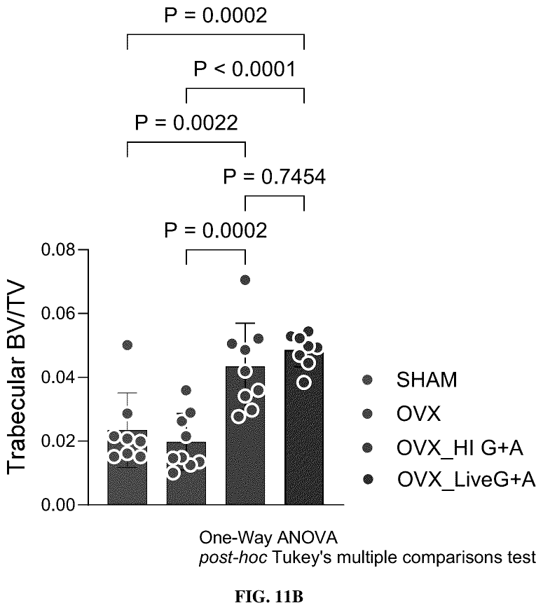

Alendronate and HI P. goldsteinii, either alone or in combination corrected ovariectomy induced decreases in stiffness in femur metaphysis in in vivo experiments. Synergistic effects of alendronate and HI P. goldsteinii in reverting ovariectomy-induced bone (femur metaphysis) weakening revealed through biomechanical analysis of Elastic energy and Stiffness. Results are shown in FIG. 10.

Although, when administered individually, no changes in elastic energy of femurs in ovariectomized mice were observed due to administration of alendronate or HI P. goldsteinii, as compared to control mice. However, co-administration of alendronate and HI P. goldsteinii not only rescued mice from loss of elastic energy in femurs after ovariectomy, but this improvement was observed to be statistically beyond even the control mice that were not ovariectomized. These results are consistent with a synergistic improvement in elastic energy of femurs in the mouse model of post-menopausal osteoporosis. Results are shown in FIG. 10

Without exception, the combined therapy of alendronate and HI P. goldsteinii was observed to be superior as compared to individual treatments in the curative experiment for all parameters assessed by the 3-point bending test. Consistent with experiments involving warm microbiota (Chevalier et al., 2020), the effects were observed to be more pronounced in femur metaphysis than femur midshaft. These results were consistent with data from preventative experiments involving administration of alendronate and/or HI P. goldsteinii prior to ovariectomy and onset of symptoms.

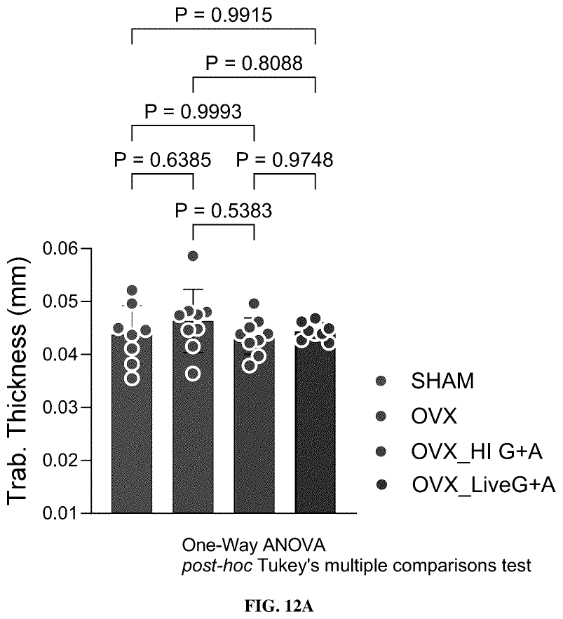

Additional experiments were performed to assess the combination or either live P. goldsteinii or HI P. goldsteinii in combination with alendronate. Increased trabecular bone volume (FIG. 11A) and bone volume to total volume ratio (FIG. 11B) of femur in mice treated with either HI or Live Parabacteroides goldsteinii (1.5 OD/ml, 200 μl per mouse every second day) and alendronate (80 μg/kg BW, once a week) starting 7-10 days after the ovariectomy for 2 months. Ovariectomies are done on 16 weeks old mice. Notably, the increases were observed to be more prominent in combination of HI or live P. goldsteinii with Alendronate than sham controls, and both live and HI Parabacteroides goldsteinii in combination with Alendronate showed higher values higher than any previous data we have with ovariectomized mice that were individually-treated.

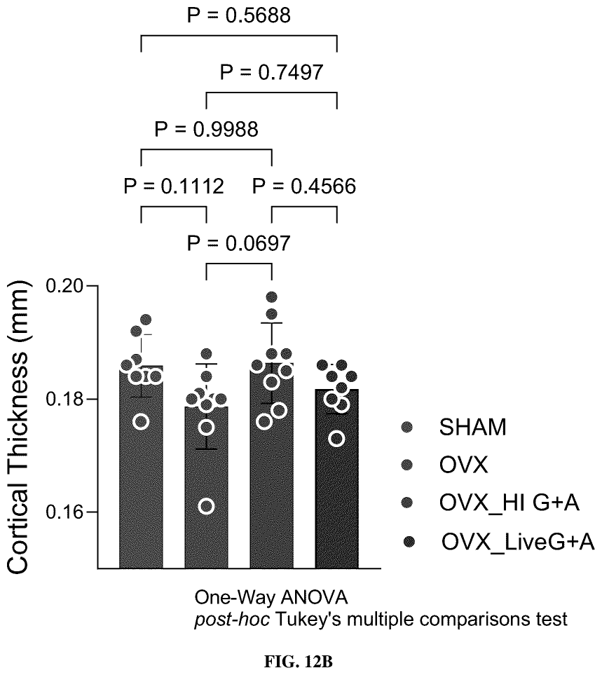

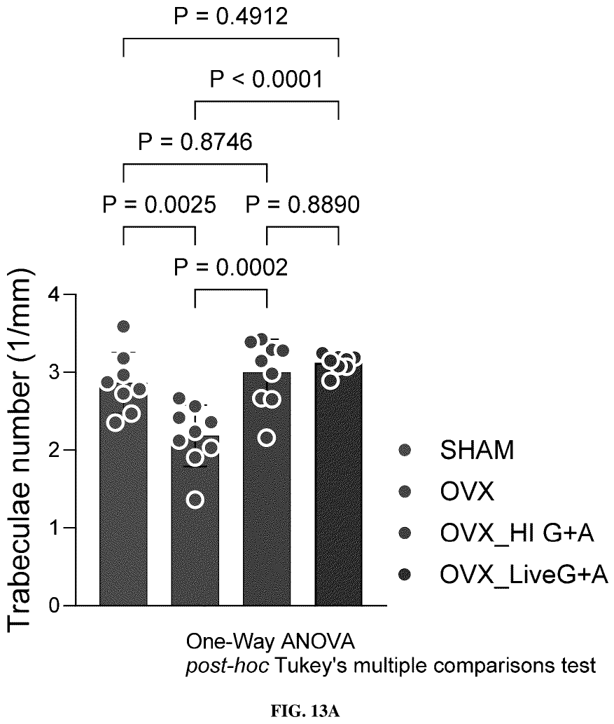

HI Parabacteroides goldsteinii and alendronate prevented ovariectomy-induced reduction of cortical thickness in the femur. While no statistical differences were detected in Trabecular thickness (FIG. 12A), cortical thickness of femur (FIG. 12B) was increased especially in mice treated with HI Parabacteroides goldsteinii (1.5 OD/ml, 200 μl per mouse every second day) and Alendronate (80 μg/kg BW, once a week) starting 7-10 days after the ovariectomy for 2 months. Ovariectomies are done on 16 weeks old mice. Notably, in all panels, there the ovariectomized mice treated with the combination therapy have values similar to the healthy, cham operated controls.

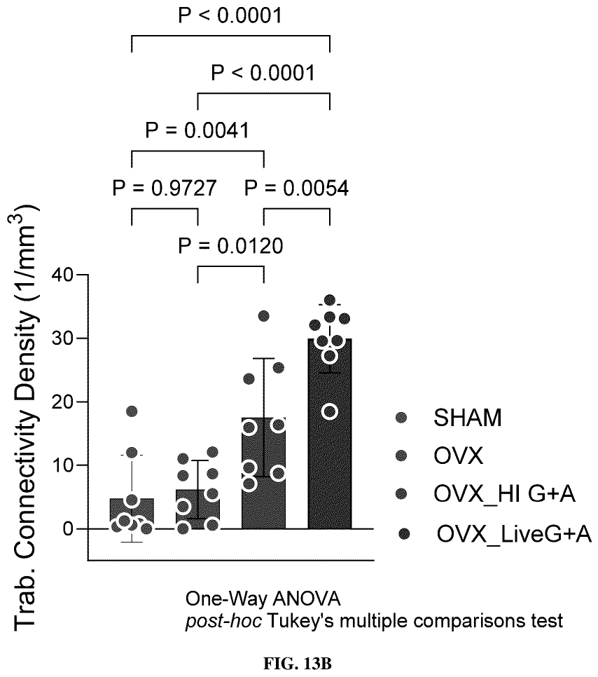

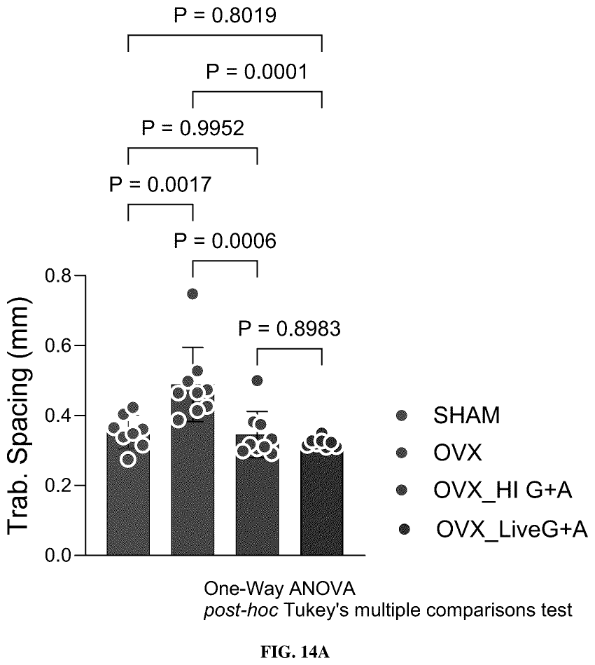

Both of either HI P. goldsteinii or live P. goldsteinii in combination with alendronate prevented the ovariectomy-induced reduction in trabecular number (FIG. 13A) and increased trabecular connectivity density (FIG. 13B) of femur. Mice were treated either with HI P. goldsteinii or live P. goldsteinii (1.5 OD/ml, 200 μl per mouse every second day) in combination with alendronate (80 μg/kg BW, once a week) starting 7-10 days after the ovariectomy for 2 months. Ovariectomies were performed on 16-week-old mice. Notably, in (FIG. 13A) the ovariectomized mice treated with the combination therapy were observed to have values similar to the healthy, cham operated controls, while in (FIG. 13B), the trabecular connectivity density exceeded the controls.

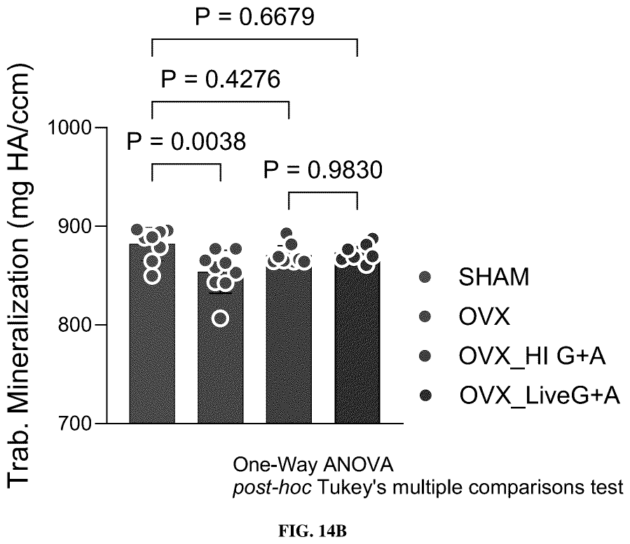

Both HI P. goldsteinii or live P. goldsteinii in combination with alendronate prevented the ovariectomy-induced increase in trabecular spacing (FIG. 14A) and reduction in trabecular mineralization (FIG. 14B). Mice were treated either with HI or Live Parabacteroides goldsteinii (1.5 OD/ml, 200 μl per mouse every second day) and alendronate (80 μg/kg BW, once a week) starting 7-10 days after the ovariectomy for 2 months. Ovariectomies performed on 16 weeks old mice. As shown in FIGS. 14A-B, the ovariectomized mice administered the combination were observed to have values similar to the healthy, sham operated controls.

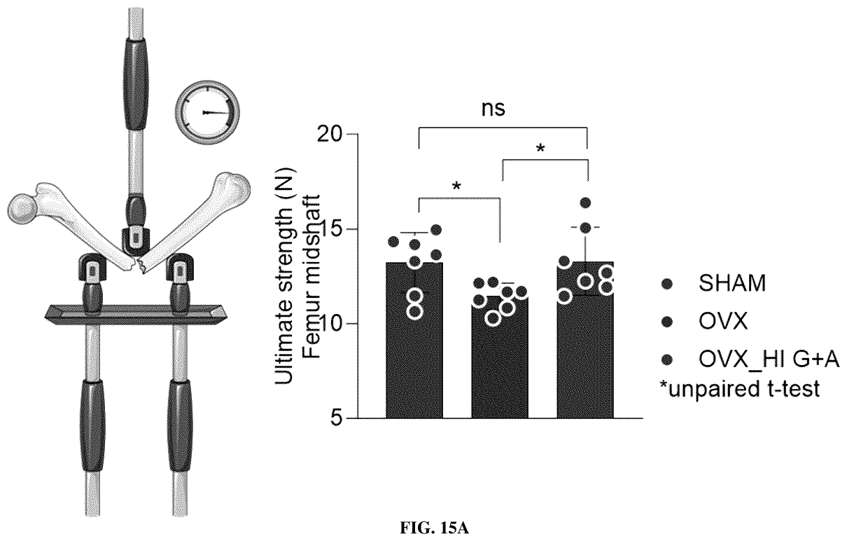

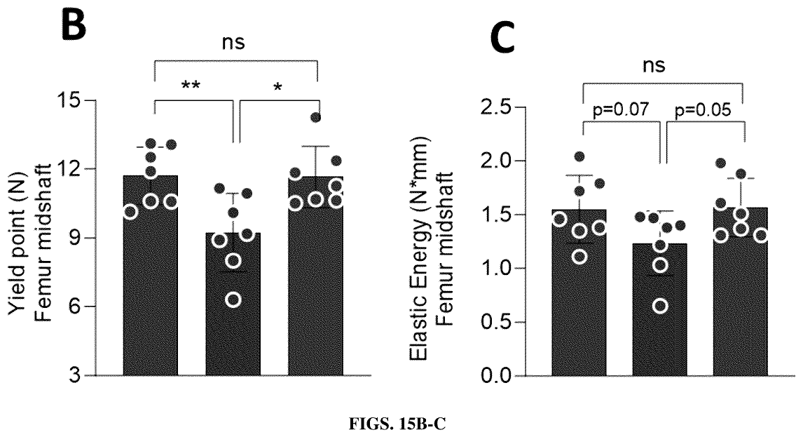

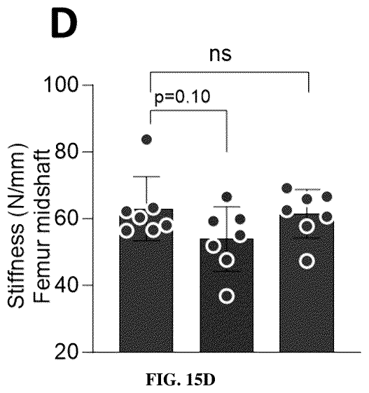

HI Parabacteroides goldsteinii and alendronate prevented the ovariectomy-induced reduction of femur strength addressed using a 3-point bending test. This combination fully prevented the ovariectomy-induced reduction in the ultimate strength (FIG. 15A), yield point (FIG. 15B), elastic energy (FIG. 15C) and stiffness (FIG. 15D) of femur midshaft. Mice were treated with HI Parabacteroides goldsteinii (1.5 OD/ml, 200 μl per mouse every second day) and Alendronate (80 μg/kg BW, once a week) starting 7-10 days after the ovariectomy for 2 months. Ovariectomies were done on 16 weeks old mice.

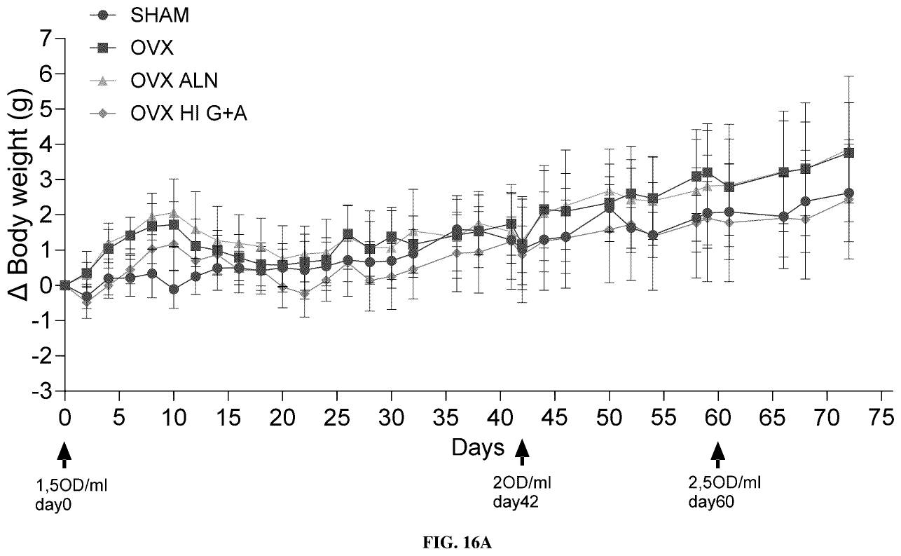

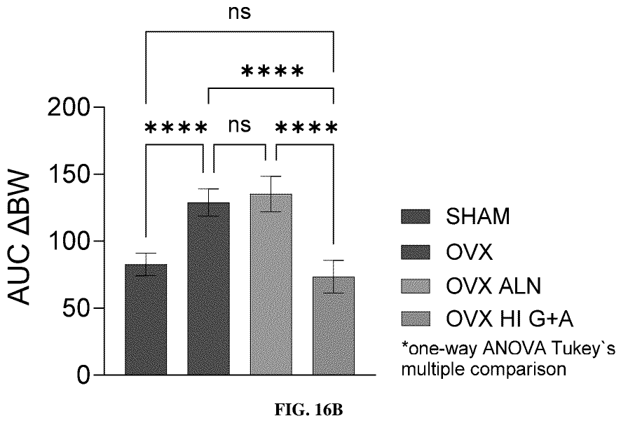

HI Parabacteroides goldsteinii and alendronate were observed to prevent the ovariectomy-induced increase in body weight, represented as change in the body weight relative to day 0 (start of the treatment, FIG. 16A), or corresponding area under the curve (FIG. 16B). Mice were treated with HI Parabacteroides goldsteinii with increasing bacterial doses (1.5-2.5 ODml, 200 μl per mouse every second day) and alendronate (80 μg/kg BW, once a week) starting 7-10 days after the ovariectomy for 2.5 months. The change in bacterial dose is indicated with arrows at the days of supplementation. Ovariectomies were performed on 16-weeks-old mice.

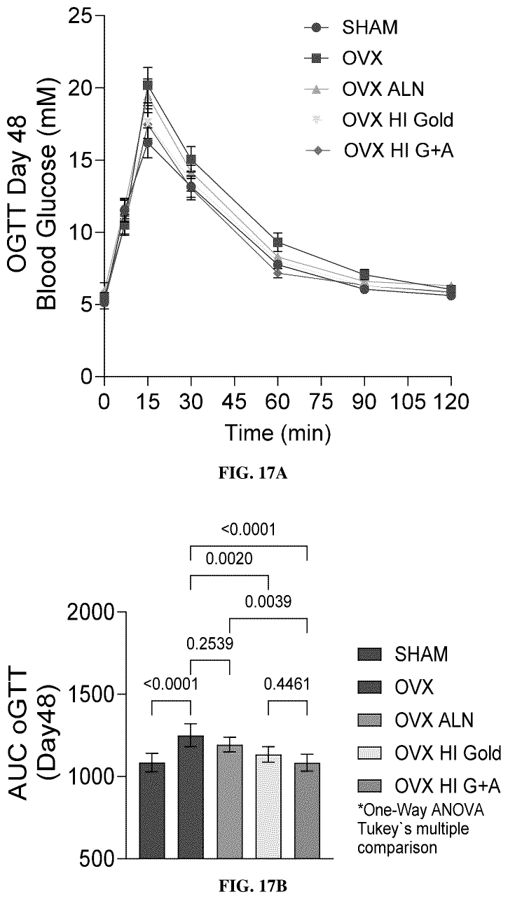

HI Parabacteroides goldsteinii and alendronate prevented ovariectomy-induced decreases in glucose tolerance (FIG. 17A), or corresponding area under the curve (FIG. 17B). Mice were treated with HI Parabacteroides goldsteinii with increasing bacterial doses (1.5-2.5 ODml, 200 μl per mouse every second day) and alendronate (80 μg/kg BW, once a week) starting 7-10 days after the ovariectomy for 2.5 months. Ovariectomies were performed on 16 weeks old mice. Notably, unlike alendronate, the HI Parabacteroides goldsteinii administration alone improved glucose tolerance (P=0.002), and this effect is potentiated in combination with alendronate (P<0.0001).

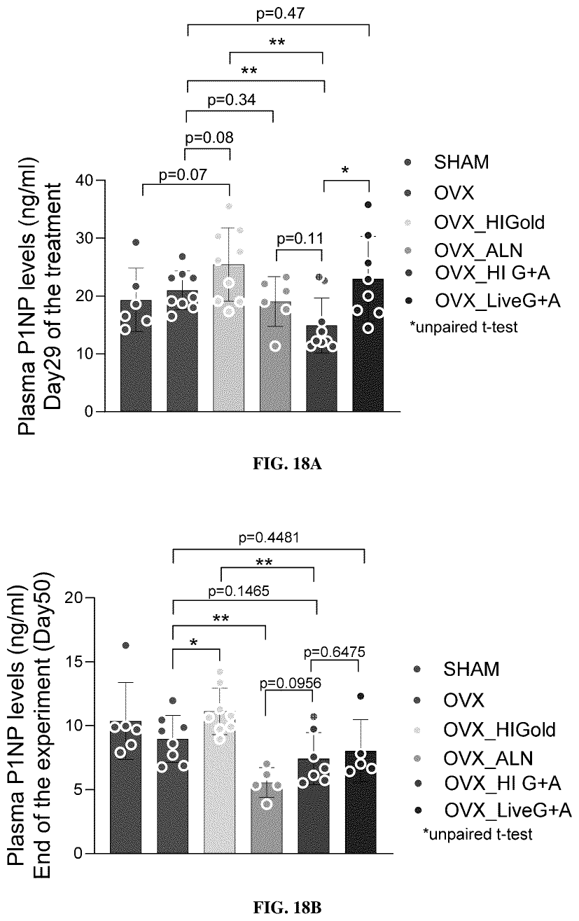

Supplementation of P. goldsteinii increases secretion the bone formation marker P1NP, measured at Day 29 (FIG. 18A), or Day 50 (FIG. 18B) after the beginning of the treatment. Bisphosponates (e.g., alendronate) prevent bone resorption, which then leads to a compensatory decrease in bone formation, since resorption and formation are coupled (Jensen et. al, Bone, 2021; Shimizu et. al, J Dent Res, 2012; Iwata et. al, Bone, 2006). Both HI and live Parabacteroides goldsteinii (1.5 OD/ml, 200 μl per mouse every second day) prevented the alendronate (80 μg/kg BW, once a week)-induced reduction in P1NP levels after 50 days of treatment. Live Parabacteroides goldsteinii were observed to exert a stronger effects than HI after 29 days of treatment, although the effects are similar after 50 days of treatment.

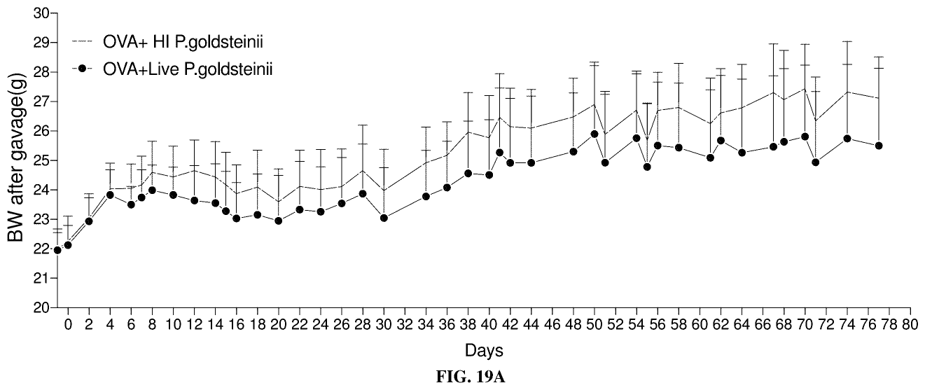

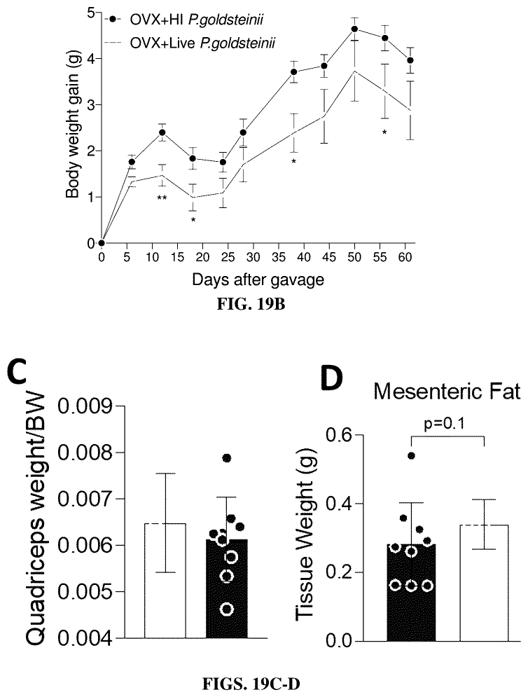

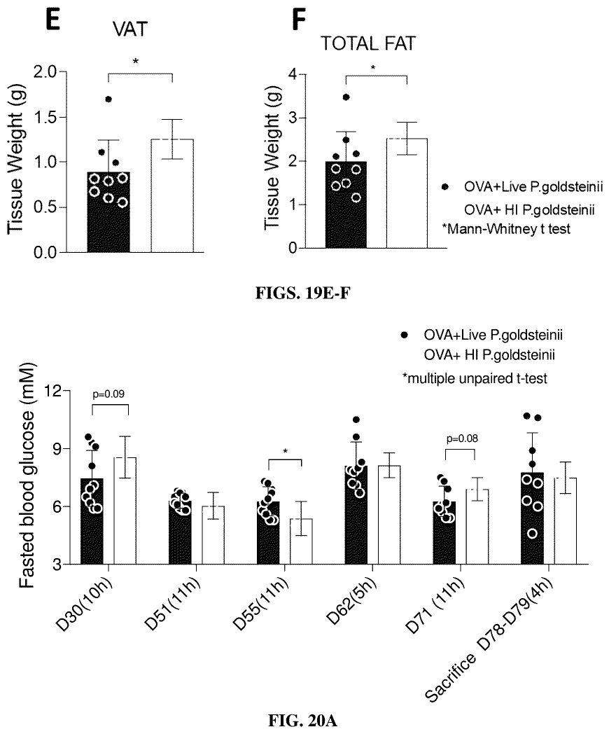

Experiments to directly compare the anti-obesogenic effects of HI and live P. goldsteinii were performed. The live P. goldsteinii were observed to exhibit slightly more prominent anti-obesogenic effects compared to HI P. goldsteinii in ovariectomized mice, primarily due to a reduction of the visceral adipose tissue (FIGS. 19A-F). Mice were treated either with HI or Live P. goldsteinii with increasing bacterial doses (1.5-2.5 OD/ml, 200 μl per mouse every second day) starting 7-10 days after the ovariectomy over 2.5 months. Ovariectomies were performed on 16-week-old mice.

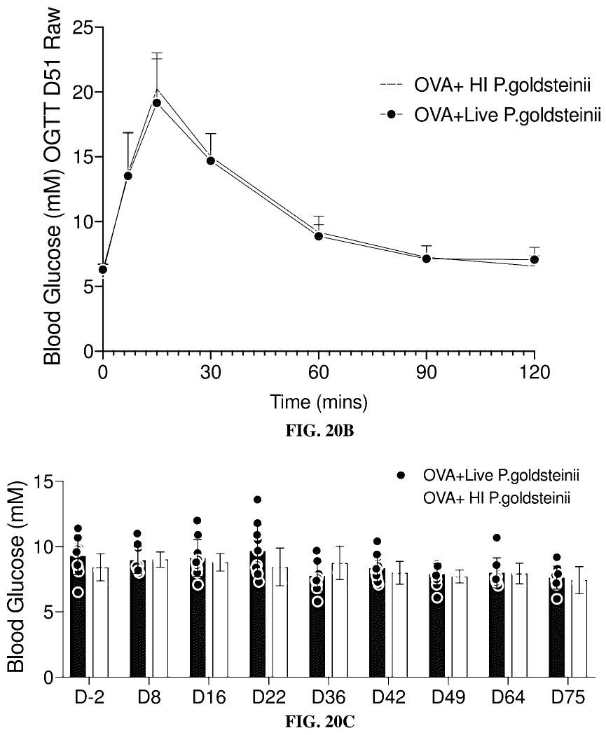

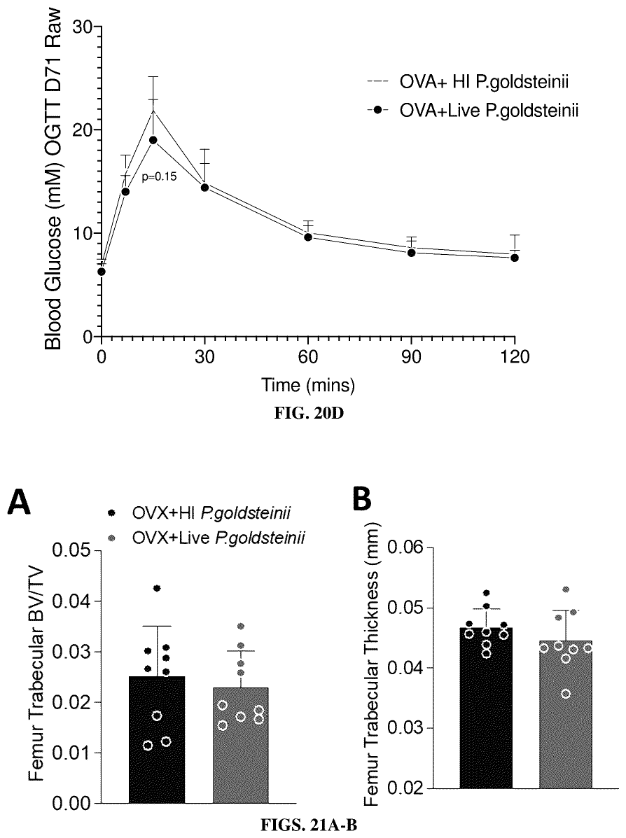

Experiments to directly compare the anti-diabetic effects of HI and live P. goldsteinii were performed. HI and live Parabacteroides goldsteinii were observed to exert similar anti-glycemic effects and glucose tolerance in ovariectomized mice, mainly due to reduction of the visceral adipose tissue (FIGS. 20A-D). Mice were treated either with HI or Live Parabacteroides goldsteinii with increasing bacterial doses (1.5-2.5 OD/ml, 200 μl per mouse every second day) starting 7-10 days after the ovariectomy over 2.5 months. Ovariectomies were performed on 16-week-old mice.

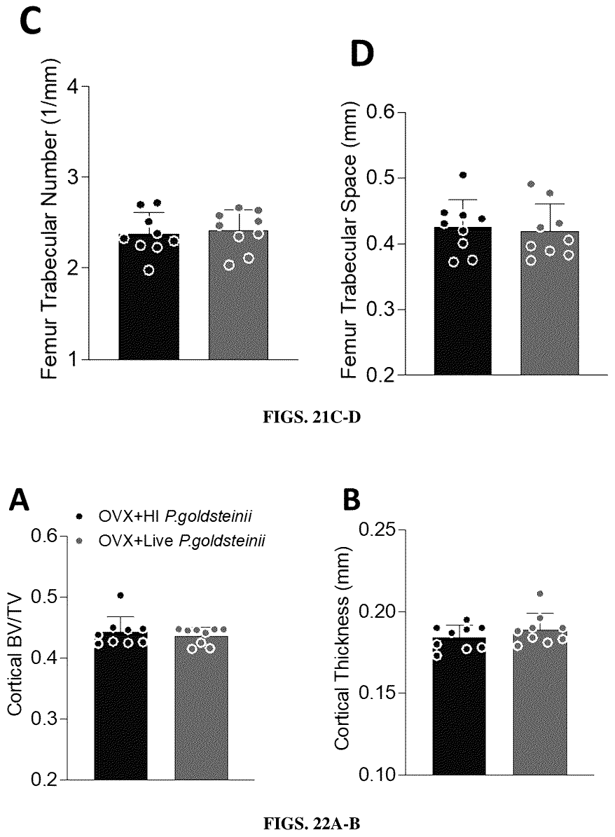

Experiments to directly compare the anti-osteoporotic effects (as measured in trabecular bone) of HI and live P. goldsteinii were performed. HI and live P. goldsteinii were observed to exert similar effects on the Trabecular bone volume (FIG. 21A), Trabecular thickness (FIG. 21B), Trabecular number (FIG. 21C) and Trabecular space (FIG. 21D) in femur of ovariectomized mice. Mice were treated either with HI or Live Parabacteroides goldsteinii with increasing bacterial doses (1.5-2.5 OD/ml, 200 μl per mouse every second day) starting 7-10 days after the ovariectomy over 2.5 months. Ovariectomies were performed on 16-week-old mice.

Experiments to directly compare the anti-osteoporotic effects (as measured in cortical bone) of HI and live P. goldsteinii were performed. HI and live P. goldsteinii exerted similar effects on the cortical bone volume (FIG. 22A) and Cortical thickness (FIG. 22B) in femur of ovariectomized mice. Mice were treated either with HI or live P. goldsteinii at increasing bacterial doses (1.5-2.5 OD/ml, 200 μl per mouse every second day) starting 7-10 days after the ovariectomy over 2.5 months. Ovariectomies were performed on 16-week-old mice.

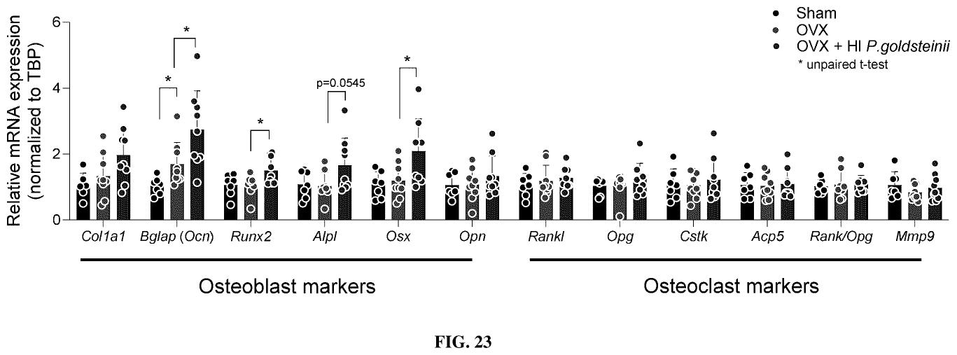

HI P. goldsteinii increased mRNA expression of osteoblast markers in tibias. Without wishing to be bound by any theory, these results support the idea that HI P. goldsteinii can exert biological effects due to acting on or affecting the function of osteoblasts. Mice were treated with HI P. goldsteinii (1.5-2 OD/ml, 200 μl per mouse every second day) starting 7-10 days after the ovariectomy for 1 month. Ovariectomies were performed on 16 week-old-mice. Results are shown in FIG. 23.

### Example 2

### Materials and Methods

Ovariectomy: 16 weeks old C57Bl/6J female mice, obtained through Janvier, were be anesthetised with Xylazin/Ketamin (injection of 120 μl of a mixture of 120 mg/kg ketamine and 16 mg/kg xylazine) and shaved below the ribs on the back side. Betadine was be applied to the area for appropriate disinfection. After a 1-2 cm incision through the skin and the muscle layer just below the ribs, the ovary was localized, the fallopian tube ligated with dissolving suture and the ovary removed. The muscle layer was sutured with dissolving suture, the wound closed with staples and disinfected. The same procedure was/will be performed on the other side. A dose of Tamgesic was/will be administered 4 hours after the surgery, and the staples were removed 7 days after the surgery under isoflurane anesthesia. The sham-operated animals underwent the same procedure, without ligating the fallopian tube and the ovary excision. All mice were then kept for additional 2 months after the ovariectomies before starting the respective curative treatments.

Microbiota transplantation: Lactobacillus gasseri (DSM 20604) and Parabacteroides goldsteinii (DSM 19948) were pushased from DSMZ. Lactobacillus reuteri (PTA-6475) was obtained from ATCC. Lactobacillus gasseri and reuteri were grown in MRS (deMan, Rogosa and Sharpe, USbiological Life Sciences, L1021-01) medium, P. goldsteinii in anaerobe basal broth (Thermo Scientific Oxoid Microbiology Products, CM0957) in an anaerobic incubator (Coy vinyl anaerobic chamber type C) set at 37° C. with a gas mix of 5% CO2, 5% H and 90% N. P. goldsteinii was inactivated at 100° C. for 15 min before the gavage, and the inactivation was confirmed. Freshly prepared bacteria were diluted in anaerobic PBS to a final concentration equivalent to 1.5 OD/ml at 600 nm. 200 ul of this suspension was gavaged every second day to ovariectomized mice, starting 2 months post-surgery for 38 day. From day 38 of the gavage until the day od sacrifice at day 60 the concentration was equivalent to 2 OD/ml at 600 nm, of which 200 ul was gavaged.

Alendronate administration: Alendronate sodium salt was purchased from Merck (Cat. No. 126855), and the powder was dissolved in sterile PBS or 0.9% NaCl. The amount administered was 80 μg/kg body weight once per week with subcutaneous injection, in total amount of 100-200 μl per injection.

Biomechanical analysis of the bones: A 3-point bending test was used to measure biomechanical parameters of the bone. Femurs were placed on two supports separated by 9.9 mm and the load was applied to the midpoint of the shaft (creating a 3-points bending). Mechanical resistance to failure (displacement and load applied) was measured using a servo-controlled electromechanical system (Instron 1114, Instron corp., High Wycombe, UK) with actuator displaced at 2 mm/minute. Ultimate force (maximal load, measured in Newtons [N]), Yield point (N), stiffness (elastic energy, N/mm), and energy to fracture (surface under the curve of the plastic region, N*mm) were calculated. Young's modulus (MPa) was determined by the equation previously described (Chevalier et. al., 2020).

Metabolic Experiments: Oral glucose tolerance tests (OGTT) were performed after 10 hr overnight fasting by oral gavage of glucose bolus (2 mg/kg body weight). Insulin tolerance test was performed after a 5 hr fasting started early in the morning, with an intraperitoneal injection of 0.5 U/kg (I9278, Sigma-Aldrich).

Measuring plasma bone turnover markers: After 5 h fasting (during the treatment), blood samples were collected from the tail of the mice with microvette capillary tubes containing EDTA and plasma was isolated after centrifuging 10000 g for 10 minutes. Plasma samples are collected during sacrifice (at the end of experiment) by taking 500 μl of blood from terminally anesthetized mice in tubes with 10 μl of 0.5 mM EDTA, 4 μl of aprotinin (1.3%) and 4 μl of DPP-IV (10 mM), followed by centrifugation as described before. N-terminal pro-collagen type 1 extension pro-peptide (PINP) was measured with an ELISA kit (cat no:AC-33F, Immunodiagnostic Systems) according to manufacturers' instructions.

Bone microarchitecture evaluation by micro-CT: Frozen femurs from the left leg that were kept at 0.9% NaCl were thawed, scanned with high-resolution microtomography (PCT, VivaCT40/Scanco system; Zurich, Switzerland), and analyzed Scanco Medical microCT software via the following the guidelines⁸³. First, femur length was measured before scanning to define the parts of the bone. Then, images were obtained at 55 keV energy and 145 μA current for 12.5 m cubic resolution. To assess trabecular bone microarchitecture, 100 slices starting from 50 slices below the distal growth plate were analyzed in the femoral metaphysis region. The cortical bone of the femur was analyzed at the level of mid-diaphysis via the adaptive-iterative threshold approach that is described in our previous study (Chevalier et. al., 2020). Three-dimensional reconstructions were generated with the following parameters: Sigma: 1.5, Support: 2, Threshold: 230-300 (spongiosa, manually decided according to image), and Sigma: 0.8, Support: 1, Threshold: 340-390 (cortex, manually decided according to image). The analyzed structural parameters for trabecular bone are bone volume fraction (BV/TV), trabecular number (1/mm), trabecular thickness (mm), trabecular space (mm), connectivity density (1/mm³). The analyzed structural parameters for cortical bone are bone volume fraction (BV/TV), cortical thickness (mm), cortical bone volume (BV, mm³), tissue mineral density (mg HA/cm³).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
PCT/US20/51581
U.S. patent Ser. No. 11/541,083
Abreu-Vieira et al., Molecular Metabolism, 4(6): p. 461-470, 2015.
Alhilli and Wright, British Journal of Experimental Pathology, 64(1): p. 34-42, 1983.
Ashoub, M. A., Nature, 181(4604): p. 284, 1958.
Bianchi et al., Health Qual Life Outcomes, 3: p. 78, 2005.
Bray et al., Endocr Rev.; 39(2): 79-132, April 2018.
Britton et al., Journal of Cellular Physiology, 2014. 229(11): p. 1822-1830, 2014.
Calinescu et al., Eur J Pharm Biopharm., 60(1):53-60, 2005.
Caporaso et al., Isme Journal, 6(8): p. 1621-1624, 2012.
Caporaso et al., Proceedings of the National Academy of Sciences of the United States of America, 108: p. 4516-4522, 2011.
Chelakkot et al., Experimental & Molecular Medicine volume 50, pagee 450, 2018.
Chevalier et al., Cell Metabolism, 32(4): p. 575-90, 2020.
Chevalier et al., Cold. Cell, 163(6): p. 1360-74, 2015.
Choi et al., Sci Rep, 5: 15878, 2015.
Cui et al., Physiol Rep, 4(10), 2016.
Dar et al., Bone Rep, 8: p. 46-56, 2018.
Davis, Nutr Today; 51(4): 167-174, July-August 2016.
de Bakker et al., JBiomech, 2018.
deMan et al. USbiological Life Sciences, L1021-01.
Depommier et al. Nat Med. 25(7):1096-1103, 2019.
Derrien et al., Int J Syst Evol Microbiol, 54: 1469-1476, 2004.
Devlin et al., Journal of Bone and Mineral Research, 2010. 25(9): p. 2078-2088, 2010.
Drake et al., Mayo Clin Proc., 83(9): 1032-1045, 2008.
Eisenberg et al., Nature Medicine, 22(12):1428-1438, 2016.
Ellis and Kuehn, Microbiol Mol Biol Rev., 74: 81-94, 2010.
Erttmann et al., Nat Commun 10:3493, 2019.
Fabbiano et al., Cell Metab, 24(3): p. 434-46, 2016.
Fabbiano et al., Cell metabolism, In press, 2018.
Finnegan et al., Journal of Antimicrobial Chemotherapy, 65:2108-2115, 2010.
Govander et al., AAPS PharmSciTech, 15(1):29-43, 2014.
Grigoryan et al., Journal of Hygienic Engineering and Design, UDC 579.67:1-11.
Harland, S. C., Nature, 186(4723): p. 446-446, 1960.
Hong et al., Allergy, 66: 351-359, 2011.
Horstman and Kuehn, J Biol Chem., 277: 32538-32545, 2002.
Hussan et al., Journal of Pharmacy 2(6):5-11, 2012.
Igarashi et al., Int J Biochem Cell Biol., 42(1):39-51, 2010.
Iwaniec and Turner, J Endocrinol, 230(3): p. R115-30, 2016.
Iwasa et al., Horm Behav, 97: p. 25-30, 2018.
Iwata et al. Bone; 39(5):1053-1058, 2006.
Jensen et al. Circulation.; 129(25 Suppl 2):S102-38; 40, Jun. 24 2014.
Jensen et al. Bone. ; 145:115850, 2021.
Kaiyala et al., Plos One, 7(8), 2012.
Kang et al., PLoS ONE, 8: e76520, 2013.
Kim et al., Clin Exp Allergy, 43: 443-454, 2013.
Kuehn and Kesty, Genes Dev., 19: 2645-2655, 2005.
Lee et al. Proteomics, 7: 3143-3153, 2007.
Lee et al., Proteomic, 9: 5425-5436., 2009.
Legroux-Gerot et al., Calcified Tissue International, 81(3): p. 174-182, 2007.
Li, et al., Journal of Clinical Investigation, 126(6): p. 2049-2063, 2016.
Lin et al., Microbiol Methods, 112:3-10, 2015.
Maclean et al., Appl Environ Microbiol, 75(7):1932-7, 2009.
Malik et al., J. Food Eng., 114(3):391-396, 2013.
Manas, et al., Journal of Applied Microbiology 98:1387-1399, 2005.
Mcmillan et al., Calcified Tissue International, 44(6): p. 399-405, 1989.
Meyer et al., Frontiers in Physiology, 8, 2017.
Million et al. Int J Obes (Lond), 36(6): 817-825, June 2012.
Morrison et al., Neurosci Lett., 197(1):5-8, 1995.
Nguyen, Sci Rep. 6: 36707, 2016.
Nilsson et al., J Intern Med, 2018.
Ohlsson and Sjogren, Trends Endocrinol Metab, 26(2): p. 69-74, 2015.
Paschen et al., 1987.
Reginster and Burlet, Bone, 38(2): p. 4-9, 2006.
Romsos, et al., Metabolism-Clinical and Experimental, 34(10): p. 931-937, 1985.
Sanderson et al., Journals of Gerontology Series a-Biological Sciences and Medical Sciences, 52(1): p. B20-B25, 1997.
Serrat, et al., Journal of Orthopaedic Research, 33(5): p. 692-698, 2015.
Serrat, et al., Proceedings of the National Academy of Sciences of the United States of America, 105(49): p. 19348-19353. 2008.
Shimizu et al., J Dent Res; 91(3):268-74, 2012.
Sjogren et al., J Bone Miner Res, 27(6): p. 1357-67, 2012.
Soda et al., J Immunol., 175(1):237-45, 2005.
Sozen et al., European Journal of Rheumatology, 4(1): p. 46-56, 2017.
Suarez-Zamorano et al., Nat Med, 2015. 21(12): p. 1497-1501, 2015.
Tian et al., J Biomed Res. 24(4):264-9, 2010.
Turchanowa et al., Eur J Clin Invest. 30(1):72-8, 2000.
Vernon et al., Aliment Pharmacol Ther.;34(3):274-85, August 2011.
Villareal Arch Intern Med, 166(22): p. 2502-10, 2006.
Wallia et al., JAMA.;311(22):2315-25, Jun. 11 2014.
Wilson et al. Front Cell Infect Microbiol. 9: 2, 2019.
World Health Organ Tech Rep Ser.; 894( ):i-xii, 1-253; PMID: 11234459, 2000.
Worthmann Nat Med, 23(7): p. 839-849, 2017.
Wright, 1983.
Wu et al., Hirsutella sinensis, Gut. 68(2):248-262, 2019.
Yamamoto et al., Br J Pharmacol., 166(3): 1084-1096, 2012.
Yang et al., 235(1-2):1-15, 2002.
Zhang et al. s, Evid Based Complement Alternat Med., 2022.
Zietak Cell Metabolism, 23(6): p. 1216-1223, 2016.

What is claimed is:

1. A method of treating a disease in a mammalian subject, comprising administering to the mammalian subject a therapeutically relevant amount of:
(i) a composition comprising a Parabacteroides goldsteinii, wherein the composition is delivered to the gastrointestinal system of the mammalian subject, and
(ii) a bisphosphonate;
wherein the disease is metabolic disease or disorder or a bone disease.

2. The method of claim 1, wherein the bisphosphonate is alendronate, risedronate, ibandronate, zoledronic acid, denosumab, raloxifene, or bazedoxifene.

3. The method of claim 1, wherein the bisphosphonate is alendronate.

4. The method of claim 3, wherein about 10-80 mg of alendronate per week or about 1-15 mg of alendronate per day is administered to the subject.

5. The method of claim 4, wherein about 35-70 mg per week of alendronate or about 5-10 mg per day of alendronate is administered to the subject.

6. The method of claim 4, wherein less than 35 mg per week of alendronate or less than 5 mg per day of alendronate is administered to the subject.

7. The method of claim 4, wherein about 10-30 mg per week of alendronate or about 1-4 mg per day of alendronate is administered to the subject.

8. The method of any one of claims 3-7, wherein the alendronate is administered orally, intravenously, intraperitoneally, or subcutaneously.

9. The method of claim 1, wherein the Parabacteroides goldsteinii is living or is not inactivated.

10. The method of claim 1, wherein the Parabacteroides goldsteinii is inactivated.

11. The method of claim 10, wherein the Parabacteroides goldsteinii is heat-inactivated.

12. The method of claim 11, wherein the Parabacteroides goldsteinii have been inactivated by heating to about 95-105° C. for about 10-20 min.

13. The method of claim 11, wherein the Parabacteroides goldsteinii have been inactivated by heating to about 100° C. for about 15 min.

14. The method of claim 1, wherein the inactivated Parabacteroides goldsteinii has been inactivated via exposure to a peroxide.

15. The method of claim 14, wherein the peroxide is hydrogen peroxide.

16. The method of claim 14, wherein the peroxide is hydrogen peroxide vapor.

17. The method of claim 1, wherein the inactivated Parabacteroides goldsteinii has been inactivated via exposure to radiation or ionizing radiation.

18. The method of claim 17, wherein the ionizing radiation comprises or consists of light having a wavelength of about 400-420 nm.

19. The method of claim 1, wherein the inactivated Parabacteroides goldsteinii has been inactivated via exposure to air plasma, ultrasound under pressure, an alcohol, high hydrostatic pressure (HHP), or pulsed electric field (PEF).

20. The method of claim 19, wherein the alcohol is ethanol.

21. The method of claim 1, wherein the composition comprises extracellular vesicles from Parabacteroides goldsteinii.

22. The method of any one of claims 1-21, wherein the composition comprises about 1×10⁸-1×10¹³ or about 1×10⁹-1×10¹⁰ cfu of the inactivated Parabacteroides goldsteinii.

23. The method of claim 22, wherein the subject is administered from about 0.25*10⁹ to about 12*10¹⁰ cells/kg body weight of the subject of the inactivated Parabacteroides goldsteinii.

24. The method of claim 23, wherein the subject is administered from about 0.97-1.62*10⁹ or about 6.8-11.3*10¹⁰ cells/kg body weight of the subject of the inactivated Parabacteroides goldsteinii.

25. The method of any one of claims 22-24, wherein the inactivated Parabacteroides goldsteinii is heat-inactivated Parabacteroides goldsteinii.

26. The method of any one of claims 22-25, wherein the inactivated Parabacteroides goldsteinii is administered to the subject once per day or once every two days.

27. The method of any one of claims 1-22, wherein the composition further comprises Lactobacillus gasseri, Lactobacillus reuteri, or Akkermansia muciniphila.

28. The composition of any one of claims 1-27, wherein the composition is further defined as a pharmaceutical composition.

29. The composition of any one of claims 1-27, wherein the composition is further defined as a probiotic composition.

30. The method of any one of claims 1-29, wherein the composition further comprises Lactobacillus gasseri and Lactobacillus reuteri.

31. The method of any one of claims 1-29, wherein the composition further comprises extracellular vesicles from Lactobacillus gasseri or Lactobacillus reuteri.

32. The method of any one of claims 1-31, wherein the pharmaceutical or probiotic composition is administered orally, colonically, via enema, via an orogastric tube, or via a nasogastric tube.

33. The method of any one of claims 1-32, wherein the inactivated Parabacteroides goldsteinii or vesicles from Parabacteroides goldsteinii is comprised in a pharmaceutical or probiotic composition that is resistant to degradation in the stomach but releases bacteria in the small intestine and/or large intestine of the subject.

34. The method of any one of claims 1-33, wherein the pharmaceutical or probiotic composition comprises an enteric coating, chitosan-alginate beads, or a hydrogel.

35. The method of claim 34, wherein the enteric coating is a fatty acid, a wax, a shellac, a plastic such as a phthalate, CAP, CAT, PVAP, HPMCP, or a plant fiber.

36. The method of any one of claims 1-33, wherein the pharmaceutical or probiotic composition does not comprise an enteric coating.

37. The method of any one of claims 1-36, wherein the pharmaceutical or probiotic composition is a tablet or capsule.

38. The method of any one of claims 1-37, wherein the subject is a human.

39. The method of claim 38, wherein the human is a postmenopausal woman.

40. The method of any one of claims 1-39, wherein the metabolic disease or disorder is obesity, type 2 diabetes, fatty liver disease, glucose intolerance, insulin resistance, post-menopausal weight gain, post-menopausal glucose intolerance, or dyslipidemia.

41. The method of claim 40, wherein the metabolic disease or disorder is obesity.

42. The method of claim 40, wherein the metabolic disease or disorder is fatty liver disease.

43. The method of claim 40, wherein the fatty liver disease is nonalcoholic fatty liver disease (NAFLD).

44. The method of any one of claims 1-39 or 41-43, wherein the subject does not have diabetes.

45. The method of any one of claims 1-39, wherein the bone disease is osteoporosis, osteomalacia, osteolysis, osteochondrodysplasias, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, or Paget's disease.

46. The method of claim 45, wherein the bone disease is osteoporosis.

47. The method of any one of claims 1-43, wherein the method further comprises administering an estrogen therapy to the subject.

48. The method of any one of claims 1-43, wherein the microbiota in the composition has been purified or cultured.

49. The method of any one of claims 1-48, wherein the method further comprises enterically administering spermine and/or spermidine to the subject.

50. The method of claim 49, wherein the method comprises enterically administering both spermine and spermidine to the subject.

51. The method of claim 49, wherein the method comprises administering about 1-50 mg per kg body weight per day spermine to the subject.

52. The method of claim 49, wherein the method comprises administering about 1-50 mg per kg body weight per day spermidine to the subject.

53. The method of any one of claims 1-52, wherein the composition comprises the spermine and/or spermidine.

54. The method of claim 53, wherein the composition comprises both spermine and spermidine.

55. The method of any one of claims 1-54, wherein Parabacteroides goldsteinii are cultured or expanded in a medium comprising spermidine or spermine prior to inactivation.

56. The method of claim 55, wherein the medium comprises about 0.1-6 mM spermidine.

57. The method of claim 55, wherein the medium comprises about 0.1-6 mM spermine.

58. The method of any one of claims 1-57, wherein the subject is administered antibiotics and exposed to an environment of about 25-50° C., more preferably about 32-35° C. for at least about 15 minutes.

59. A pharmaceutical or probiotic composition comprising:
(i) Parabacteroides goldsteinii, the growth medium of Parabacteroides goldsteinii, or vesicles from Parabacteroides goldsteinii, and
(ii) a biphosphate;
wherein the composition is formulated for delivery to the gastrointestinal system.

60. The composition of claim 59, wherein the Parabacteroides goldsteinii is inactivated.

61. The composition of claim 59, wherein the Parabacteroides goldsteinii is living or is not inactivated.

62. The composition of any one of claims 59-61, wherein the biphosphate is alendronate.

63. The composition of claim 62, wherein the composition comprises about 1-75 mg of alendronate.

64. The composition of claim 62, wherein the composition comprises about 5-70 mg of alendronate.

65. The composition of claim 62, wherein the composition comprises less than 5 mg or about 1-4 mg of alendronate.

66. The composition of any one of claims 59-65, wherein the composition comprises about 1×10⁸-1×10¹³ or about 1×10⁹-1×10¹⁰ cfu of the inactivated Parabacteroides goldsteinii

67. The composition of claim 66, wherein the composition comprises 6.5-11.5*10¹⁰ cells of heat-inactivated Parabacteroides goldsteinii.

68. The composition of any of claims 66-67, wherein the wherein the inactivated Parabacteroides goldsteinii is heat-inactivated Parabacteroides goldsteinii.

69. The composition of any one of claims 59-67, wherein the composition further comprises Lactobacillus gasseri or Lactobacillus reuteri.

70. The composition of any one of claims 59-67, wherein the composition further comprises extracellular vesicles from Lactobacillus gasseri or extracellular vesicles from Lactobacillus reuteri.

71. The composition of any one of claims 59-70, wherein the pharmaceutical or probiotic composition is formulated for oral, colonic, enema, orogastric, or nasogastric administration.

72. The composition of any one of claims 59-71, wherein the pharmaceutical or probiotic composition is resistant to degradation in the stomach but releases bacteria in the small intestine and/or large intestine of the subject.

73. The composition of claim 72 wherein the pharmaceutical or probiotic composition comprises an enteric coating, chitosan-alginate beads, or a hydrogel.

74. The composition of claim 73, wherein the enteric coating is a fatty acid, a wax, a shellac, a plastic such as a phthalate, CAP, CAT, PVAP, HPMCP, or a plant fiber.

75. The composition of claim 72 wherein the pharmaceutical or probiotic composition does not comprise an enteric coating.

76. The composition of any one of claims 59-75, wherein the pharmaceutical or probiotic composition is a tablet or capsule.

77. The composition of any one of claims 59-76, wherein the pharmaceutical or probiotic composition further comprises spermine or spermidine.

78. The composition of claim 77, wherein the pharmaceutical or probiotic composition comprises 1-3500 mg of spermine.

79. The composition of claim 77, wherein the pharmaceutical or probiotic composition comprises 1-3500 mg of spermidine.

80. The composition of any one of claims 77-79, wherein the pharmaceutical or probiotic composition comprises both spermine and spermidine.

81. The composition of any of claims 59-80, wherein the Parabacteroides goldsteinii has been inactivated via exposure to a peroxide, ionizing radiation, heat, air plasma, ultrasound under pressure, an alcohol, high hydrostatic pressure (HHP), or pulsed electric field (PEF).

82. The composition of claim 81, wherein the Parabacteroides goldsteinii has been inactivated via exposure to a peroxide, ionizing radiation, or heat.

83. The composition of any one of claims 59-82, wherein the composition for use in treating a metabolic disease or disorder in a mammalian subject.

84. The composition of claim 83, wherein the metabolic disease or disorder is obesity, type 2 diabetes, fatty liver disease, a nonalcoholic fatty liver disease (NAFLD), insulin resistance, or dyslipidemia.

85. The composition of claim 84, wherein the subject is a human.

86. The composition of claim 85, wherein the human is a postmenopausal woman.
